(19) United States
(12) Patent Application Publication
(10) Pub. No.: US 2003/0134790 A1
(43) Pub. Date: Jul. 17, 2003

(54) Bone Morphogenetic Protein-2 And Bone Morphogenetic Protein-4 In The Treatment And Diagnosis Of Cancer

(75) Inventor: John Langenfeld, Flemington, NJ (US)

Correspondence Address: PERKINS COIE LLP, POST OFFICE BOX 1208, SEATTLE, WA, 98111-1208, US

(73) Assignee: University of Medicine and Dentistry of New Jersey

(21) Appl. No.: 10/139,814

(22) Filed: May 2, 2002

Related U.S. Application Data
(63) Continuation-in-part of application No. 10/044,716, filed on Jan. 11, 2002.

(51) Int. Cl. A61K 38/17; A61K 38/43

(52) U.S. Cl. 514/12

(57) ABSTRACT

The present invention pertains to the use of BMP-2 and/or BMP-4 as 1) targets for cancer treatment therapies and 2) means to diagnose cancer. The therapeutic component of this invention involves administering to a patient a composition that inhibits bone morphogenetic protein-2 activity and/or bone morphogenetic protein-4 activity. Such inhibition may be accomplished by ligands or antibodies that bind to BMP-2 and/or BMP-4 or receptors for BMP-2 and BMP-4. It may also be achieved by preventing the processing of pro-BMP-2 and/or pro-BMP-4, or blocking transcription or replication of BMP-2 DNA and/or BMP-4 DNA or translation of BMP-2 mRNA and/or BMP-4 mRNA. The diagnostic component of the invention involves measuring the BMP-2 and/or BMP-4 level(s) in biological samples from both a patient and a non-cancerous subject and comparing those levels. Elevated levels of BMP-2 and/or BMP-4 in the patient compared to the subject indicate cancer.

This application claims the benefit of U.S. application Ser. No. 10/044,716 (Langenfeld), filed Jan. 11, 2002. This application also incorporates by reference U.S. application Ser. No. 10/044,716.

### FIELD OF USE

The present invention relates to the fields of molecular biology, immunology, and medicine and provides methods for the treatment and diagnosis of cancer. Specifically, it relates to the use of bone morphogenetic protein-2 (BMP-2) and bone morphogenetic protein-4 (BMP-4) as 1) targets for cancer treatment therapies and 2) means to diagnose cancer.

### BACKGROUND OF THE INVENTION

Various publications or patents are referred to in parentheses throughout this application. Each of these publications or patents is incorporated by reference herein. Complete citations of the scientific publications referred to in this section, the Background of the Invention, are set forth at the end of this section. All other citations are set forth in the text.

Lung cancer is the leading cause of cancer deaths in the United States with over 150,000 people this year expected to die from this disease (1). Despite improvements in diagnosis and treatment, only 10% of lung cancer patients survive 5 years (1) with the majority of patients succumbing due to spread of the tumor to other parts of the body. The genes that induce the invasion and metastasis of lung cancers are poorly understood. Applicant's experiments to identify genes that regulate metastasis revealed that bone morphogenetic protein-2 (BMP-2) is overexpressed in human lung carcinomas. Subsequent experiments revealed that BMP-2 is also overexpressed in many other common human cancers. Applicant also found gene expression of BMP-4, a protein that is highly homologous to BMP-2, in human lung cancer tumor samples.

BMP-2 and BMP-4 are powerful morphogenetic proteins that have been studied predominantly for their role in embryonic development and their ability to induce bone formation. The bone morphogenetic proteins (BMPs) are members of the transforming growth factor (TGF) superfamily, which are a phytogenetically conserved group of proteins (2). There are 20 isotypes of the BMPs, with BMP-2 and BMP-4, which share 92% homology, placed in the same subclass based on their similar structures. (3, 4, 5). BMP-2 and BMP-4 are secreted proteins that induce pluripotential mesenchymal differentiation (6, 7) (8) and are required for the normal embryonic development of many organs including lung and bone (9, 10). BMP-2 and BMP-4 also have chemotactic properties capable of inducing the migration of normal vascular endothelial and mononuclear cells (12, 13).

The BMPs are synthesized as inactive variable length precursor proteins (14, 15). The precursor BMP-2 and BMP-4 proteins are proteolytically cleaved, producing mature C-terminal proteins of a little more than 100 residues (4, 11, 14). BMP-2 and BMP-4 interact with the same binding sites: mature BMP-2 and BMP-4 protein signaling is mediated by transmembrane serine/threonine kinases called type IA, IB, and type II receptors (4, 16-20). The receptor phosphorylates cytoplasmic targets, which includes the Smad family of proteins (21). In addition, the same molecules, including noggin, chordin, DAN, gremlin, and cerberus 1 homolog, inhibit both BMP-2 and BMP-4, thereby preventing their ability to bind to the receptors. (22-24)

While BMP expression has been noted in a few cancers, such as sarcomas (25) and pancreatic cancer (26) and in cancer cell lines (27), the inhibition of BMP-2 activity and/or BMP-4 activity as a potential cancer treatment has neither been mentioned nor studied in the literature. To the contrary, several articles suggest that BMP-2 and/or BMP-4 have an inhibitory effect on cancer cell proliferation and may be useful therapeutic agents to treat cancer. (28, 29, 30, 31)

Applicant has discovered that expression of bone morphogenetic protein-2 (BMP-2) is linked to cancer invasion and growth and that inhibiting BMP-2 activity reduces the size of cancerous tumors in nude mice and down regulates the expression of VEGF and sonic hedgehog in lung cancer cell lines. Applicant has also discovered BMP-4 gene expression in human lung cancer tumors. As discussed above, BMP-2 and BMP-4 share very similar structures and nearly identical biological activity. Thus, the present invention is directed toward using BMP-2 and/or BMP-4 as targets for cancer treatment therapies and as a means to diagnose cancer. Specifically, the therapeutic component of this invention involves administering to a patient a composition that inhibits bone morphogenetic protein-2 activity and/or bone morphogenetic protein-4 activity. The diagnostic component of the invention involves measuring the BMP-2 and/or BMP-4 level(s) in biological samples from both a patient and a non-cancerous subject and comparing those levels, with elevated levels indicating cancer in the patient.

References

1. Thoracic Surgery, 1^{st} ed., vol. 1 (Churchill Livingstone, N.Y., Edinburgh, London, Melbourne, Tokyo) (1995).

2. Warburton, D., et al. “The molecular basis of lung morphogenesis” Mech Dev 92, 55-81 (2000).

3. Celeste A J, et al. “Identification of transforming growth factor beta family members present in bone-inductive protein purified from bovine bone” Proc Natl Acad Sci USA 87, 9843-9847 (1990).

4. Leong, L. M., et al., “Bone Morphogenetic Protein-4” Int. J. Biochem. Cell Biol. 28, 1293-96 (1996).

5. Zimmerman, L. B., et al. “The Spemann Organizer Signal Noggin Binds and Inactivates Bone Morphogenetic Protein 4” Cell 86, 599-606 (1996).

6. Erickson, D. M., et al. “Recombinant bone morphogenetic protein (BMP-2) regulates costochondral growth plate chondrocytes and induces expression of BMP-2 and BMP-4 in a cell maturation-dependent manner” J Orthop Res 15, 371-80 (1997).

7. An, J., Rosen, et al. “Recombinant human bone morphogenetic protein-2 induces a hematopoietic microenvironment in the rat that supports the growth of stem cells” Exp Hematol 24, 768-75 (1996).

8. Abe, E., et al. “Essential requirement of BMPs-2/4 for both osteoblast and osteoclast formation in murine bone marrow cultures from adult mice: antagonism by noggin” J Bone Miner Res 15, 663-73 (2000).

9. Vainio, S., et al. “Identification of BMP-4 as a signal mediating secondary induction between epithelial and mesenchymal tissues during early tooth development” Cell 75, 45-58 (1993).

10. Weaver, M., et al. “BMP signaling regulates proximal-distal differentiation of endoderm in mouse lung development” Development 126, 4005-15 (1999).

11. Wozney, J. M., et al. “Novel regulators of bone formation: molecular clones and activities” Science 242, 1528-34 (1988).

12. Willette, R. N., et al. “BMP-2 gene expression and effects on human vascular smooth muscle cells” J Vasc Res 36, 120-5 (1999).

13. Cunningham, N. S., et al. “Osteogenin and recombinant bone morphogenetic protein 2B are chemotactic for human monocytes and stimulate transforming growth factor beta 1 mRNA expression” Proc Natl Acad Sci USA 89, 11740-4 (1992).

14. Cui, Y., et al. “BMP-4 is proteolytically activated by furin and/or PC6 during vertebrate embryonic development” Embo J 17, 4735-43 (1998).

15. Sugiura “Cloning and functional characterization of the 5′-flanking region of the human bone morphogenetic protein-2 gene” Biochem J 3338, 443-440 (1999).

16. Koenig, B. B., et al. “Characterization and Cloning of a Receptor for BMP-2 and BMP-4 form NIH 3T3 Cells” Molecular and Cellular Biology 14, 5961-74 (1994).

17. Liu, F., et al. “Human type II receptor for bone morphogenetic proteins (BMPs): extension of the two-kinase receptor model to the BMPs” Mol Cell Biol 15, 3479-86 (1995).

18. Ikeda, T., et al. “Cloning of rat type I receptor cDNA for bone morphogenetic protein-2 and bone morphogenetic protein-4, and the localization compared with that of the ligands” Dev Dyn 206, 318-29 (1996).

19. ten Dijke, P., et al. “Activin receptor-like kinases: a novel subclass of cell-surface receptors with predicted serine/threonine kinase activity” Oncogene 8, 2879-87 (1993).

20. ten Dijke, P., et al. “Identification of type I receptors for osteogenic protein-1 and bone morphogenetic protein-4” J Biol Chem 269, 16985-8 (1994).

21. Sakou, T., et al. “Localization of Smads, the TGF-beta family intracellular signaling components during endochondral ossification” J Bone Miner Res 14, 1145-52 (1999).

22. Piccolo, S., et al. “Dorsoventral patterning in Xenopus: Inhibition of ventral signals by direct binding of chordin to BMP-4” Cell 86, 589-98 (1996).

23. Piek, E., et al. “Specificity, diversity, and regulation in TGF-β superfamily signaling” The FASEB Journal 13, 2105-24 (1999).

24. McMahon, J. A., et al. “Noggin-mediated antagonism of BMP signaling is required for growth and patterning of the neural tube and somite” Genes & Development 12, 1438-52 (1998).

25. Guo, W., et al. “Expression of bone morphogenetic proteins and receptors in sarcomas” Clin. Orthop. 365: 175-83 (1999).

26. Kleef, J., “Bone Morphogenetic Protein-2 exerts diverse effects on cell growth in vitro and is expressed in human pancreatic cancer in vivo” Gastroenterology 116: 1202-1216 (1999).

27. Hatakeyama, S., et al., “Expression of bone morphogenetic proteins of human neoplastic epithelial cells” Biochem Mol. Biol. Int. 42(3): 497 (1997)

28. Hjertner, O., et al., “Bone morphogenetic protein-4 inhibits proliferation and induces apoptosis of multiple myeloma cells” Blood 97(2): 516-22 (2001).

29. Kawamura, C., et al., “Bone morphogenetic protein-2 induces apoptosis in human myeloma cells with modulation of STAT3” Blood 96(6): 2005-11 (2000).

30. Soda, H. “Antiproliferative effects of recombinant human bone morphogenetic protein-2 on human tumor colony-forming units” Anticancer Drugs 9(4): 327-31 (1998).

31. Tada, A., et al., “Bone morphogenetic protein-2 suppresses the transformed phenotype and restores actin microfilaments of human lung carcinoma A549 cells” Oncol. Rep. 5(5): 137-40 (1998).

### SUMMARY OF THE INVENTION

The present invention is related to Applicant's discovery that bone morphogenetic protein-2 (BMP-2) is overexpressed in many common human cancers and is linked to cancer invasion and growth. Further, inhibiting BMP-2 activity reduces the size of cancerous tumors in nude mice and down regulates the expression of VEGF and sonic hedgehog in lung cancer cell lines. In addition, gene expression of BMP-4 was detected in cancerous human lung tumors. As discussed above, BMP-4 is highly homologous to BMP-2 and shares almost identical biological activity with BMP-2. Thus, the present invention pertains to the use of BMP-2 and/or BMP-4 as 1) targets for cancer treatment therapies and 2) means to diagnose cancer.

A primary aspect of the present invention is to provide a method for the treatment of cancer by administering to a patient a therapeutically effective amount of a BMP-2 and/or BMP-4 activity inhibitor. Some cancers that may be treated by this method are carcinomas, including, but not limited to, lung cancer, bladder cancer, breast cancer, colon cancer, kidney cancer, ovarian cancer, thyroid cancer, endometrial cancer, omental cancer, testicular cancer, and liver cancer. In a preferred embodiment of this invention the patient is human.

The BMP-2 inhibitor of this invention may be a polypeptide that binds specifically to bone morphogenetic protein-2, a polypeptide that binds specifically to a BMP-2 receptor, or an antibody that binds specifically to BMP-2. The BMP-2 inhibitor may also be an antisense oligonucleotide that binds to a BMP-2 nucleic acid sequence or some portion thereof.

The BMP-4 inhibitor of this invention may be a polypeptide that binds specifically to bone morphogenetic protein-4, a polypeptide that binds specifically to a BMP-4 receptor, or an antibody that binds specifically to BMP-4. The BMP-4 inhibitor may also be an antisense oligonucleotide that binds to a BMP-4 nucleic acid sequence or some portion thereof.

This invention features several particular polypeptides that inhibit BMP-2 and/or BMP-4. Preferred embodiments of this invention feature known antagonists to BMP-2 and BMP-4, such as noggin, chordin, cerberus 1 homolog, gremlin, and DAN. Noggin is particularly preferred. Another aspect of this invention relates to the use of fragments of noggin, chordin, cerberus 1 homolog, gremlin, and DAN as BMP-2 and/or BMP-4 inhibitors.

Another embodiment of this invention provides a method for treating cancer by administering to a patient a therapeutically effective amount of an expression vector encoding a BMP-2 and/ or a BMP-4 inhibitor, such as a polypeptide that binds BMP-2 and/or BMP-4 or antisense oligonucleotides that bind to the nucleic acid for BMP-2 and/or BMP-4. Another aspect of this invention includes the expression vector described above in which the nucleic acid sequence that causes inhibition of BMP-2 and/or BMP-4 is operably linked to a selective promoter. One preferred selective promoter encompassed by this invention is carcinoembryonic antigen promoter.

This invention also encompasses a kit that includes packaging material, a BMP-2 activity inhibitor and/or a BMP-4 activity inhibitor, and instructions that indicate that the compounds can be used for treating cancer in a patient. One type of cancer that may be treated is carcinoma. Particular carcinomas encompassed by this invention are lung cancer, bladder cancer, breast cancer, colon cancer, kidney cancer, ovarian cancer, thyroid cancer, endometrial cancer, omental cancer, testicular cancer, and liver cancer.

The diagnostic component of this invention includes a method for diagnosing cancer in a patient by obtaining a biological sample from a patient and measuring the level of BMP-2 and/or BMP-4 in the biological sample, with an elevated level or elevated levels of BMP-2 and/or BMP-4 indicating cancer in the patient.

Any assay available to measure BMP-2 and/or BMP-4 levels is encompassed by this invention. Particularly preferred are immunoassays. Some examples of immunoassays included in this invention are Enzyme-Linked Immunosorbent Assay (ELISA), Western blot, immunoprecipitation, in situ immunohistochemistry, and immunofluorescence. The Enzyme-Linked Immunosorbent Assay is most particularly preferred.

Another aspect of this invention is a method for the diagnosis of cancer in a patient by detecting overexpression of BMP-2 and/or BMP-4 in the patient by (i) quantifying in vivo or in vitro the presence of BMP-2 and/or BMP-4 in a patient or a biological sample obtained from a patient, (ii) comparing the result obtained in step (i) to that of a normal, non-cancerous patient, and (iii) diagnosing for the presence of cancer based on an increased level of BMP-2 and/or BMP-4 in step (ii) relative to a normal, non-cancerous patient.

### BRIEF DESCRIPTION OF THE FIGURES

FIG. 1 illustrates representational difference analysis (RDA) subtraction. FIG. 1(a) shows amplification of CDNA prior to subtraction. Lane 1: IHBE cells; lane 2: lung carcinoma. FIG. 1(b) shows the distinct CDNA bands present after the second round of subtraction and amplification. FIG. 1(c) lists the proteins that were identified by a BLAST data base search after the DNA corresponding to each of the bands shown in FIG. 1(b) was isolated and sequenced.

FIG. 2 is an ethidium-stained agarose gel showing the results of RT-PCR performed on human lung cancer specimens. Lanes 1-4 contain the results of the RT-PCR of various specimens, while lane 5 contains a marker.

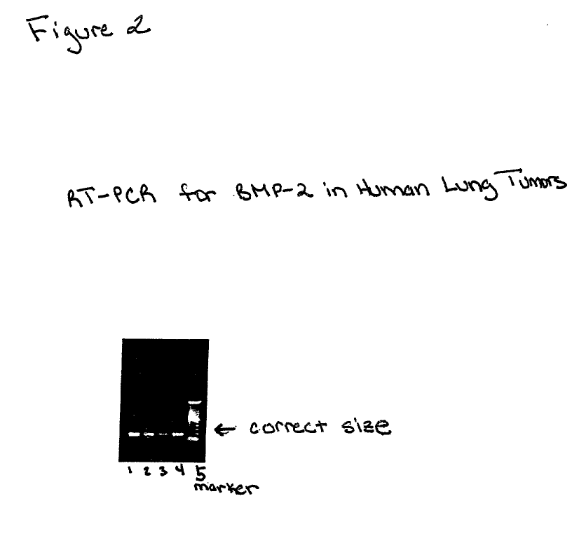

FIG. 3 illustrates Western blots showing mature BMP-2 overexpressed in lung cancer tissue specimens and lung cancer cell lines. FIG. 3(a) is a representative Western blot showing overexpression of BMP-2 in cancer tissue specimens. Lanes 1-5: normal lung tissue, lane 6: SOAS osteosarcoma cell line, lanes 7-11: non-small lung cell carcinomas. FIG. 3(b) is the corresponding actin immunoblot. FIG. 3(c) is a Western blot of non small cell lung carcinoma (NSCLC) subtypes. Lanes 1-4: normal lung tissue, lane 5: squamous carcinoma, lane 6: adenocarcinoma, lane 7: bronchoalveolar carcinoma, lane 8: large cell carcinoma. FIG. 3(d) is the corresponding actin immunoblot. FIG. 3(e) is a BMP-2 immunoblot of lane 1: benign lung tumor, lane 2: mesthotheleoma, lane 3: normal lung tissue, lane 4: carcinoid tumor, lane 5: normal lung, lane 6: NSCLC, lane 7: normal lung tissue, lane 8: NSCLC, lane 9: recombinant BMP-4. FIG. 3(f) is a BMP-4-probed Western blot with the same lane contents as FIG. 3(e), except lane 9, which is recombinant BMP-4. FIG. 3(g) is the corresponding actin immunoblot.

FIG. 4(a) is a Western immunoblot of total cellular protein that demonstrates that normal and malignant human lung cell lines express mature BMP-2 protein. Lanes (1) IHBE; (2) SOAS; (3) H7249; (4) A549. (b) Western blot of cell culture media shows lung cancer cell lines secrete a BMP-2 precursor protein. Lanes (1) lung cancer tumor specimen; (2) A549 media; (3) H7249 media; (4) MHBE; (5), NBE media; (6) serum free media alone. (c) immunoblot of BMP type IA receptor. Lanes (1-3) normal lung tissue specimens; (4) IHBE cells; (5) H7249 cells; (6) A549 cells; (7-9) lung cancer tissue specimens. (d) immunoblot of BMP type 1B receptor. (1-3) normal lung tissue specimens; (4) IHBE cells; (5)H7249 cells; (6) A549 cells; (7-9) lung cancer tissue specimens.

FIG. 5: 5(a) is an immunohistochemistry localizing BMP-2 expression to the tumor cells. BMP-2 expression in a NSCLC demonstrating cytoplasmic staining of the tumor cells (arrowheads). The nuclei (n) of the tumor cells and the interstitium (I) are non-reactive; (b) Preabsorption of the BMP-2 antibody with recombinant human BMP-2 is non-reactive with the tumor cells (arrows). Original magnification ×82.

FIG. 6(a) is a BMP-2 Western blot of human breast tumors and corresponding normal tissue. Lane 1: NSCLC, lane 2-5: breast carcinomas, lane 6-8: normal breast tissue, lane 9: recombinant BMP-2. FIG. 6(b) is a BMP-2 Western blot of common human carcinomas and the corresponding normal tissue. Lane 1: normal endometrium, lane 2: endometrial carcinoma, lane 3: ovarian carcinoma, lane 4: normal colon, lane 5: colon carcinoma, lane 6: normal bladder, lane 7: bladder carcinoma.

FIG. 7(a) is a Western blot showing BMP-2 expression in metastatic tumors. Lane 1: interstitial inflammatory lung disease, lane 2: normal omentum, lane 3: metastatic kidney tumor, lane 4: normal lymph node, lane 5: metastatic breast cancer, lane 6: metastatic kidney tumor, lane 7: metastatic NSCLC, lane 8: omentum carcinoma. FIG. 7(b) is the corresponding actin immunoblot. FIG. 7(c) is a BMPR IA Western blot, while FIG. 7(d) is a BMPR IB Western blot. The contents of the lanes on both blots are the same: lane 1: normal kidney, lanes 2-3: normal lung, lane 4: metastatic kidney carcinoma, lane 5: metastatic breast carcinoma, lane 6: metastatic NSCLC, lanes 7-9: NSCLC. FIG. 7(e) is BMPR IA Western blot and FIG. 7(f) is a BMPR IB Western blot of common human carcinomas. Lane contents are the same on both blots: lane 1: normal kidney, lane 2: normal endometrium, lane 3: omentum, lane 4: normal colon, lane 5: ovarian carcinoma, lane 6: kidney carcinoma, lane 7: endometrial carcinoma, lane 8: omenental Stumor, lane 9: colon carcinoma.

FIG. 8 is a Western blot showing BMP-2 in serum samples from lung cancer patients. Lanes 1-2: serum samples, lane 3: recombinant BMP-2.

FIG. 9 shows that secreted BMP-2 precursor is proteolytically cleaved by human leukocytes. Cell culture media from the A549 cells incubated with leukocytes for 16 hours is probed with BMP-2 antibody recognizing its mature C-terminal end. FIG. 9(a) is the resulting Western blot: lane 1: A549 lysate, lane 2: media without leukocytes, lanes 3-4: media with human leukocytes. FIG. 9(b) is the same immunoblot hybridized with BMP-2 antibody recognizing its N-terminal end. FIG. 9(c) is a Western blot of leukocyte samples probed with anti-furin antibody.

FIG. 10 shows that BMP-2 treatment enhances formation of blood vessels around a cancerous tumor. Each picture is of tissue from a nude mouse injected either with A549 cells or with A549 cells and BMP-2. The picture in the upper right shows tissue (including a tumor) from a nude mouse injected with A549 cells. Upper left: control. Upper right: mouse treated with BMP-2. Lower left: mouse treated with noggin.

FIG. 11 shows tissue (from nude mice injected with A549 cells and nude mice co-injected with A549 cells and BMP-2) stained with anti-CD 31 antibody, which recognizes endothelial cells, viewed from under a microscope. Left: control. Right: BMP-2 treated.

FIG. 12 shows that BMP-2 regulates sonic hedgehog expression. The Western blot on the left was probed with anti sonic hedgehog and shows an increase in sonic hedgehog expression as the amount of recombinant BMP-2 added to the A549 cell culture is increased. The Western blot on the right was probed with anti sonic hedgehog and shows A549 cell culture media without added noggin (Lane 1) and cell culture media with added noggin (Lane 2).

FIG. 13 shows that BMP-2 stimulates the migration of A549 and H7249 human lung cancer cell lines. 13(a): Recombinant human BMP-2, 1 ng/ml, 10 ng/ml, 100 ng/ml, 500 ng/ml, or 1000 ng/ml was added to the lower well of the transwell chamber. Migrated cells counted using fluorescent microscopy. 13(b) Noggin inhibits BMP-2 induced migration. Lane (1), media alone; (2) recombinant BMP2 (500 ng/ml); (3) noggin (10 mg/mi) and recombinant BMP-2 (500 ng/ml). 13(c) H7249 cells migrated off cover slips towards Affi-Blue agarose beads containing recombinant BMP-2. 13(d) H7249 cells did not migrate off cover slips toward AffiBlue agarose beads containing dilution buffer. Similar results were found using the A549 cells. All the above experiments were repeated at least 3 times. Data presented as mean+standard deviation. 13(e): Recombinant human BMP-2 stimulates the invasion of A549 or H7249 cells. Recombinant BMP-2, 1 ng/ml, 10 ng/ml, 100 ng/ml, 500 ng/ml, or 1000 ng/ml was added to the lower wells of a Matrigel invasion chamber. Experiments were repeated at least 3 times. Data presented as mean+5 standard deviation.

FIG. 14(a) show tumor growth after 19 days following the subcutaneous co-injection of A549 lung cancer cells into nude mice with Affi-blue agarose beads coated with (1) 100 ug/ml of albumin, (2) recombinant human BMP-2, or (3) recombinant mouse noggin.

FIG. 15 show that noggin inhibits VEGF expression in the A549 lung cancer cell line. The Western blot was probed with anti-VEGF antibody. The lane labeled with a plus was cell culture media from cultures treated with noggin. The lane labeled with a minus was cell culture media from control cultures.

### DETAILED DESCRIPTION OF THE INVENTION

The present invention is related to Applicant's discovery that the overexpression of bone morphogenetic protein-2 (BMP-2) is linked to cancer invasion and growth. BMP-2 is overexpressed in many common human cancers and regulates molecular pathways that are involved in the promotion of cancer. Inhibiting BMP-2 activity reduces the size of cancerous tumors in nude mice and down regulates the expression of VEGF and sonic hedgehog, which have been linked to cancer, in lung cancer cell lines. In addition, BMP-4 gene expression was detected in human lung cancer tumor specimens. BMP-4 is highly homologous to BMP-2, it is inhibited by the same molecules that inhibit BMP-2, and it binds the same receptors as does BMP-2. See Piek, E., et al. “Specificity, diversity, and regulation in TGF-β superfamily signaling” The FASEB Journal 13, 2105-24 (1999); Leong, L. M., et al., “Bone Morphogenetic Protein-4” Int. J. Biochem. Cell Biol. 28, 1293-96 (1996); Zimmerman, L. B., et al. “The Spemann Organizer Signal Noggin Binds and Inactivates Bone Morphogenetic Protein 4” Cell 86, 599-606 (1996); Piccolo, S., et al. “Dorsoventral patterning in Xenopus: Inhibition of ventral signals by direct binding of chordin to BMP-4” Cell 86, 589-98 (1996). Thus, the present invention is directed toward using BMP-2 and/or BMP-4 as a target for cancer treatment therapies and as a means to diagnose cancer.

The therapeutic component of this invention involves administering to a patient a composition that inhibits bone morphogenetic protein-2 activity and/or bone morphogenetic protein-4 activity. Such inhibition may be accomplished by ligands or antibodies that bind to BMP-2 and/or BMP-4 or receptors for BMP-2 and/or BMP-4. It may also be achieved by preventing the processing of pro-BMP-2 and/or pro-BMP-4, or blocking transcription or replication of BMP-2 DNA and/or BMP-4 DNA or translation of BMP-2 MRNA and/or BMP-4 MRNA. Delivery of such compositions may be systemic or tissue-targeted.

The diagnostic component of the invention involves measuring the BMP-2 and/or BMP-4(s) level in biological samples from both a patient and a non-cancerous subject and comparing those levels. Elevated levels of BMP-2 and/or BMP-4 in the patient compared to the subject indicate cancer.

Although specific embodiments of the present invention will now be described, it should be understood that such embodiments are examples that are merely illustrative of a small number of the many possible specific embodiments that can represent applications of the principles of the present invention. Various modifications obvious to one skilled in the art to which the present invention pertains are within the spirit, scope and contemplation of the present invention as further defined in the appended claims.

Definitions

A “bone morphogenetic protein-2 activity inhibitor” is a composition that antagonizes the activity of the BMP-2 protein by specifically binding to it or to BMP receptors, blocks the activation of pro-BMP-2, or prevents the replication or transcription of the BMP-2 gene or the translation of BMP-2 MRNA into protein.

A “bone morphogenetic protein-4 activity inhibitor” is a composition that antagonizes the activity of the BMP-4 protein by specifically binding to it or to BMP receptors, blocks the activation of pro-BMP-4, or prevents the replication or transcription of the BMP-4 gene or the translation of BMP-4 MRNA into protein.

“Polypeptide” refers to any peptide or protein comprising two or more amino acids joined to each other by peptide bonds or modified peptide bonds. Polypeptide refers to both short chains, commonly referred to as peptides, oligopeptides or oligomers, and to longer chains, generally referred to as proteins. Polypeptides include amino acid sequences modified either by natural processes, such as posttranslational processing, or by chemical modification techniques that are well known in the art. Such modifications are well described in basic texts and in more detailed monographs, as well as in a voluminous research literature.

An “expression vector” is a recombinant vector that incorporates the desired gene and associated control sequences that promote and/or regulate expression of the gene. The desired gene is “operably linked” to such control sequences. The term “operably linked” means that the regulatory sequences necessary for expression of the coding sequence are placed in an appropriate position in the expression vector relative to the coding sequence so as to enable expression of the coding sequence. The preparation of such recombinant expression vectors as well as the use of various control sequences is well known to those of skill in the art and described in many references. See, for example, Sambrook, J., et al., Molecular Cloning : A Laboratory Manual 2nd ed. (Cold Spring Harbor, N.Y., Cold Spring Harbor Laboratory) (1989).

A “selective promoter” refers to a promoter that is not indiscriminately expressed. Instead it is expressed only, for example, in certain tissues, certain tumors, in response to certain treatments, or in response to certain events in a cell. Such tissue-specific, tumor-selective, treatment-responsive, or tumor endothelium directed promoters are described in Nettlebeck, D. M., et al., “Gene therapy: designer promoters for tumour targeting” Trends Genet 16(4); 174-81 (2000).

An “expression vector vehicle”refers to an expression vector paired with a moiety that facilitates delivery of the expression construct to cells in vivo. An expression vector may incorporate genes encoding the delivery moiety. One example of such an expression vector is a viral vector.

The term “antibody” refers to polyclonal and monoclonal antibodies, chimeric, single chain, and humanized antibodies, as well as Fab fragments, including the products of a Fab or other immunoglobulin expression library.

“Polyclonal” refers to antibodies that are heterogeneous populations of antibody molecules derived from the sera of animals immunized with an antigen or antigenic functional derivative thereof. For the production of polyclonal antibodies, various host animals may be immunized by injection with the antigen. Various adjuvants may be used to increase the immunological response, especially when using an entire protein, or a larger section of the protein. The type of adjuvant used will depend on the hosts. Typical adjuvants include Fruend's, Fruend's complete, or oil-in-water emulsions. In these cases the entire protein or portion thereof can serve as the antigen. When a smaller peptide is utilized, it is advantageous to conjugate the peptide with a larger molecule to make an immunostimulatory conjugate for use as the antigen. Commonly utilized conjugate proteins that are commercially available for such use include bovine serum albumin (BSA) and keyhole limpet hemocyanin (KLH).

“Monoclonal antibodies” are substantially homogeneous populations of antibodies to a particular antigen. They may be obtained by any technique that provides for the production of antibody molecules by continuous cell lines in culture. Such methods are well known to those of ordinary skill in the art and include general hybridoma methods of Kohler and Milstein, Nature (1975) 256: 495-497, the trioma technique, the human B-cell hybridoma technique (Kozbor et al., Immunology Today 4:72 (1983) and the EBV-hybridoma technique (Cole et al., Monoclonal Antibodies and Cancer Therapy, pp.77-96, Alan R. Liss, Inc. (1985). The basic technique involves injecting a mouse, or other suitable animal, with an antigen. The animal is subsequently sacrificed and cells taken from its spleen are fused with myeloma cells. The result is a hybrid cell, referred to as a hybridoma, that reproduces in vitro. The population of hybridomas are screened to isolate individual clones each of which secrete a single antibody species to the antigen. The individual antibody species obtained in this way are each the product of a single B cell from the immune animal generated in response to a specific antigenic site recognized on the antigen. Kohler, G. and Milstein, C. Nature (London) 256: 495-497 (1975) and Eur. J. Immunol. 6: 511-519 (1976).

The term “antibody fragment” refers to a portion of an antibody, often the hyper variable region and portions of the surrounding heavy and light chains, that displays specific binding affinity for a particular molecule. The term antibody fragment also includes single chain antibodies.

An “antisense oligonucleotide” is an oligonucleotide that specifically hybridizes, under cellular conditions, with the cellular MRNA or genomic DNA encoding a BMP-2 protein and/or with the cellular mRNA or genomic DNA encoding a BMP-4 protein or some portion of such cellular or genomic DNA, thereby inhibiting biosynthesis of the BMP-2 and/or BMP-4 protein. The binding may be via conventional base pair complementarity, or, in the case of binding to DNA duplexes, via specific interactions in the major groove of the double helix.

The term “effective amount” refers to the quantity of a compound that is sufficient to yield a desired therapeutic response without undue adverse side effects (such as toxicity, irritation, or allergic response) commensurate with a reasonable benefit/risk ratio when used in the manner of this invention. The specific “effective amount” will, obviously, vary with such factors as the particular cancer being treated, the physical condition of the patient, the type of mammal being treated, the duration of the treatment, the nature of concurrent therapy (if any), and the specific formulations employed and the structure of the compounds or their derivatives.

A “patient” is a mammal suspected of having cancer. The patient is preferably human but may also be another mammal, such as a cat, dog, horse, cow, rat, or mouse.

A “biological sample” is a substance obtained from the patient's body. The particular “biological sample” selected will vary based on the cancer the patient is suspected of having and, accordingly, which biological sample is most likely to contain BMP-2 and/or BMP-4.

An “elevated level” means the level of bone morphogenetic protein-2 and/or bone morphogenetic protein-4 that is greater than the level of analyte present in a particular biological sample of patient that is not suffering from cancer.

A “carcinoma” is an epithelial cancer. Examples of carcinomas are bladder cancer, breast cancer, colon cancer, kidney cancer, lung cancer, ovarian cancer, thyroid cancer, endometrial cancer, omental cancer, testicular cancer, and liver cancer. The epithelium predominately lines ducts and lining of organs or glands.

BMP-2 and/or BMP-4 as a Target in the Treatment of Cancer

The present invention is directed to the use of BMP-2 and/or BMP-4 as a target in the treatment of cancer. Amino acids #283-396 of SEQ ID NO: 2 constitute the amino acid sequence of mature human BMP-2. Nucleotides #372-1514 of SEQ ID NO: 1 constitute the nucleotide coding sequence for human BMP-2. Amino acids #293-408 of SEQ ID NO: 18 constitute the amino acid sequence of mature human BMP-4. Nucleotides #3166-10271 of SEQ ID NO: 17 constitute the gene for human BMP-4. Exon #3 (nucleotides #7791-8167) and exon #4 (nucleotides #9131-10271) encompass the nucleotide coding sequence and 5′ and 3′ flanking regions for human BMP-4. Given the experiments described above, the 92% homology between BMP-2 and BMP-4, and their binding of the same receptors and inhibitors, any composition that 1) specifically binds BMP-2 and/or BMP-4 or a receptor for BMP-2 and/or BMP-4, thereby antagonizing BMP-2 and/or BMP-4 activity, 2) blocks the processing of pro-BMP-2 and/proBMP-2, or 3) prevents the replication or transcription of BMP-2 and/or BMP-4 DNA or the translation of BMP-2 and/or BMP-4 mRNA could be used as a therapy to treat cancer.

A compound that specifically binds to BMP-2 is any compound (such as a polypeptide or an antibody) that has a binding affinity for any naturally occurring isoform, splice variant, or polymorphism of BMP-2. As one of ordinary skill in the art will appreciate, such “specific” binding compounds may also bind to other closely related proteins that exhibit significant homology (such as greater than 90% identity, more preferably greater than 95% identity, and most preferably greater than 99% identity) with the amino acid sequence of BMP-2. Thus, a compound that specifically binds BMP-2 may also specifically bind BMP-4, to which BMP-2 is 92% homologous, thereby inhibiting BMP-4 activity as well.

A compound that specifically binds to BMP-4 is any compound (such as a polypeptide or an antibody) that has a binding affinity for any naturally occurring isoform, splice variant, or polymorphism of BMP-4. As one of ordinary skill in the art will appreciate, such “specific” binding compounds may also bind to other closely related proteins that exhibit significant homology (such as greater than 90% identity, more preferably greater than 95% identity, and most preferably greater than 99% identity) with the amino acid sequence of BMP-4. Thus, a compound that specifically binds BMP-4 may also specifically bind BMP-2, thereby inhibiting its activity as well.

Similarly, a compound that specifically binds to a BMP receptor is any compound that has a binding affinity for any naturally occurring isoform, splice variant, or polymorphism of the BMP receptor. As one of ordinary skill in the art will appreciate, such “specific” binding compounds may also bind to other closely related proteins that exhibit significant homology (such as greater than 90% identity, more preferably greater than 95% identity, and most preferably greater than 99% identity) with the amino acid sequence of a BMP receptor.

The present invention embodies polypeptides that specifically bind to BMP-2 and/or BMP-4 or that specifically bind to BMP receptors, thereby inhibiting BMP-2 and/or BMP-4 activity. Specific embodiments of such polypeptides are described below.

The present invention encompasses known antagonists of BMP-2 and BMP-4 activity, including noggin (Brunet, L. J., et al., “Noggin, Cartilage Morphogenesis, and Joint Formation in the Mammalian Skeleton” Science 280(5368): 1455-7 (1998); U.S. Pat. No. 6,075,007, Economides, et al.), chordin (U.S. Pat. No. 5,896,056, LaVallie, et al.; Millet, C., et al., “The human chordin gene encodes several differentially spliced variants with distinct BMP opposing activities” Mech. Dev. 106(1-2): 85-96 (2001)), gremlin (GenBank Accession No.

AF154054), cerberus 1 homolog (GenBank Accession No. NM_{—}005454), and DAN.

Recombinant mouse noggin from R & D Systems (Minneapolis, Minn.) was used in the inhibition experiments described in the Results section below. Mouse and human noggin share 98% homology. Therefore, this invention also relates to use of a polypeptide with the amino acid sequence of mature mouse noggin (amino acids #20-231 of SEQ ID NO: 6) and with the amino acid sequence of mature human noggin (amino acids #20-231 of SEQ ID No.: 4) as a BMP-2 activity inhibitor and a BMP-4 activity inhibitor. The amino acid sequence for human chordin is SEQ ID No: 8, for human gremlin is SEQ ID NO: 10, and for cerberus 1 homolog is SEQ ID NO: 12. The nucleotide coding sequence for human noggin is SEQ ID NO: 3, for mouse noggin is SEQ ID NO: 5, for human chordin is nucleotides #247-3114 of SEQ ID NO: 7, for human gremlin is nucleotides #130-684 of SEQ ID NO: 9, for human cerberus 1 homolog is SEQ ID NO: 11.

This invention also embodies polypeptide fragments of noggin, chordin, gremlin, cerberus 1 homolog, and DAN that bind BMP-2 and/or BMP-4, thereby inhibiting the activity of BMP-2 and/or BMP-4. Such polypeptides may be tested for inhibitory efficiency by culturing cells transformed with progressively shorter portions of the nucleotide sequences encoding the above proteins, recovering and purifying from the various cultures the resulting polypeptide, and testing those polypeptides for their ability to inhibit BMP-2 activity and/or BMP-4 activity.

This invention also includes genetically altered BMP receptor proteins that inhibit BMP-2 activity and/or BMP-4 activity. For example, altered BMP receptors that inhibit the binding effects of BMP-2 and/or BMP-4 are described in U.S. Pat. No. 6,291,206 (Wozney, et al.)

Also included by this invention are polypeptides that bind BMP receptors without activating them. (Nickel, J., et al. “The Crystal Structure of the BMP-2:BMPR-IA Complex and the Generation of BMP-2 Antagonists” The Journal of Bone & Joint Surgery 83-A, Supp.1, Part 1: 7-14 (2001). Kirsch, T., et al. “BMP-2 antagonists emerge from alterations in the low-affinity binding epitope for receptor BMPR-II” The EMBO Journal 19(13):3314-24 (2000)) Particularly preferred are ligands that will bind BMP IB receptors, as aberrant expression of the BMP IB receptor in many human cancer specimens has been noted, as discussed in the Results section below. (Ide, H., et al., “Cloning of human bone morphogenetic protein type IB receptor (BMPR-IB) and its expression in prostate cancer in comparison with other BMPRs” Oncogene 13(11): 1377-82 (1997)). The coding sequence for BMP IB precursor is nucleotides #274-1782 of SEQ ID NO: 13. The amino acid sequence for BMP IB is amino acids #14-502 of SEQ ID NO 14.

This invention also encompasses expression vectors that incorporate a nucleotide sequence encoding an inhibitor of BMP-2 activity and/or BMP-4 activity operably linked to control sequences that promote and/or regulate expression of the nucleotide sequence. The preparation of such expression vectors as well as the use of various control sequences is well known to those of skill in the art and is described in many references, such as Sambrook, et al. (1989). Expression vectors can be derived from bacterial plasmids, from bacteriophage, from transposon, from yeast episomes, from insertion elements, from yeast chromosomal elements, from viruses and from combinations thereof, such as those derived from plasmid and bacteriophage genetic elements, such as cosmids and phagemids. Promoters can be prokaryotic, such as lac, lacz, T3, T7, gpt, lambda PR, PL, and trp, or eukaryotic, such as CMV immediate early, HSV thymidine kinase, early and late SV40, LTR's from retrovirus, and mouse metallothionein-1. Selective promoters such as those described in Nettlebeck, D. M., et al., “Gene therapy: designer promoters for tumour targeting” Trends Genet 16(4); 174-81 (2000) that are tissue-specific, tumor-selective, treatment-responsive, or tumor endothelium directed may also be used. For example, the promoter of the carcinoembryonic antigen (CEA) is expressed on many breast, lung, and colorectal cancers.

For introduction of a gene that encodes a protein that antagonizes BMP-2 activity and/or BMP-4 activity an expression vector vehicle that will facilitate delivery of the desired gene to the affected cells may be used. One way to facilitate delivery is by using an expression vector derived from virus. Examples of viral vectors that have been successfully used to deliver desired sequences to cells with high infection efficiency are adenoviral, retroviral, vaccinia viral, and adeno-associated viral vectors. Commonly used viral promoters for expression vectors are derived from polyoma, cytomegalovirus, Adenovirus, and Simian Virus 40 (SV40). It is also possible to use promoter or control sequences normally associated with the desired gene sequence, if such control sequences are compatible with the host cell systems.

Non-viral expression vector vehicles are also available. For instance, the expression vector could be associated with one or more lipids. As is known in the art of lipid-based gene delivery, such nucleic acid-lipid complexes can be in a variety of different forms depending generally on the nature of the lipid employed, the ratio of nucleic acid to lipid and/or other possible components, and the method by which the complex is formed. Examples of complexes include liposomes and micelles. Liposome-mediated gene transfer seems to have great potential for certain in vivo applications in animals. Studies have shown that intravenously injected liposomes are taken up essentially in the liver and the spleen, by the macrophages of the reticuloendothelial system. Using a catheter to introduce liposomes coupled to expression vectors to particular cellular sites has also been described. (Nabel, E. G., et al., Science 249:1285-1288 (1990))

Another possible expression vector vehicle consists of a cell receptor-specific ligand and a DNA-binding agent that would bind to the expression vector. (Nishikawa, M. et al., Gene Therapy 7:548-55 (2000)). Such a vehicle could also comprise a cell receptor-specific ligand and the nucleic acid-lipid complex described above. Nicolau, C. et al., Methods Enzvmol 149: 157-76 (1987))

In addition, the present invention embodies antibodies that specifically bind BMP receptors or BMP-2 and/or BMP-4, thereby inhibiting BMP-2 activity and/or BMP-4 activity. When raising antibodies to BMP-2, BMP-4, or BMP receptors, the entire protein (either the precursor or the processed protein), or a portion thereof, may be utilized. Information usefuil in designing an antigen for the production of antibodies to BMP-2 may be deduced by those of skill in the art by homology analysis of SEQ ID NO: 2, especially amino acids #283-396 of SEQ ID NO: 2. Information useful in designing an antigen for the production of antibodies to BMP-4 may be deduced by those of skill in the art by homology analysis of SEQ ID NO: 18, especially amino acids #293-408 of SEQ ID NO: 18. Antibodies that recognize both BMP-2 and BMP-4 could be designed by one of skill in the art by analyzing the amino acid sequences of both proteins.

Recombinant human BMP-2 and BMP-4 proteins are commercially available from R & D Systems (Minneapolis, Minn.) and portions of the BMP-2 and BMP-4 proteins may be produced by a variety of methods. In order to raise antibodies to particular epitopes, peptides derived from the full BMP-2 or the full BMP-4 sequence may be used. Custom-synthesized peptides in the range of 10-20 amino acids are available from a multitude of vendors, and can be ordered conjugated to KLH or BSA. Alternatively, peptides in excess of 30 amino acids may be synthesized by solid-phase methods, or may be recombinantly produced in a recombinant protein production system. In order to ensure proper protein glycosylation and processing an animal cell system (e.g., Sf9 or other insect cells, CHO or other mammalian cells) is preferred.

Selection of antibodies which alter the activity of BMP-2 and/or BMP-4 may be accomplished in several ways. Antibodies that alter the binding of BMP-2 and/or BMP-4 to a receptor may be detected by well known binding inhibition assays. For instance, according to standard techniques, the binding of a labeled (e.g., flourescently or enzyme-labeled) antibody to BMP-2, which has been immobilized in a microtiter well, is assayed for BMP-2 binding in both the presence and absence of the appropriate receptor. The decrease in binding will be indicative of a competitive inhibitor relationship between the antibody and the receptor. The same technique could be used with BMP-4. In addition, antibodies that are useful for altering the function of BMP-2 and/or BMP-4 may be assayed in functional formats, such as the cell migration assays described in the Results and Examples sections.

This invention also embodies compositions that prevent the processing of inactive BMP-2 and/or BMP-4 precursors. BMP precursors are proteolytically activated by proprotein convertases. For example, pro-BMP-2 is cleaved by furin convertase from human leukocytes. In addition, pro-BMP-4 is cleaved by furin and/ or PC6. See Cui, Y., et al. “BMP-4 is proteolytically activated by furin and/or PC6 during vertebrate embryonic development” The EMBO Journal 17, 4735-43 (1998). Furin inhibitors are known. See, e.g., Cui, Y. et al.; Cameron, A., et al., “Polyarginines are potent furin inhibitors” J. Biol. Chem. 275: 36741-49 (2000).

While the BMP-2 and BMP-4 inhibitors discussed above adversely affect BMP-2 activity and/or BMP-4 activity after these proteins are expressed, it will be readily apparent to one of ordinary skill in the art that specific prevention of BMP-2 and/or BMP-4 biosynthesis will achieve the same goals as more direct inhibition of activity. Consequently, this invention also encompasses inhibition of BMP-2 and/or BMP-4 biosynthesis as a method for treating cancer. Such inhibition may be achieved by selectively degrading mRNA encoding BMP-2 and/or mRNA encoding BMP-4 or by interfering with transcription or translation of such mRNA. See Glavic, A., et al., “Xiro-1 controls mesoderm patterning by repressing BMP-4 expression in the Spemann organizer” Dev. Dyn. 222(3): 368-376.

Inhibition of BMP-2 and/or BMP-4 biosynthesis to treat for cancer could also be achieved through antisense therapy. Antisense therapy is the administration or in situ generation of oligonucleotides that specifically hybridize, under cellular conditions, with the cellular MRNA or genomic DNA encoding a BMP-2 or BMP-4 protein or some portion of such cellular or genomic DNA, thereby inhibiting biosynthesis of the BMP-2 or BMP-4 protein. Antisense therapy refers generally to the range of techniques known by one of ordinary skill in the art, and includes any therapy that relies on specific binding to oligonucleotide sequences.

Delivery of an antisense oligonucleotide of the present invention can occur in a variety of ways. For example, antisense oligonucleotides can be delivered as expression vectors that produces RNA which is complementary to at least a unique portion of the cellular mRNA encoding BMP-2 and/or the cellular mRNA encoding BMP-2. Such an expression vector could be delivered to cells by one of the expression vector vehicles described above. Alternatively, the antisense oligonucleotide could be generated ex vivo as an oligonucleotide probe which, when introduced to the cell, inhibits biosynthesis of BMP-2 and/or BMP-4 proteins by hybridizing with the MRNA or genomic sequences encoding BMP-2 and/or BMP-4. Such oligonucleotide probes could be modified oligonucleotides that are resistant to endogenous nucleases and therefore are stable in vivo. General methods to construct oligomers useful in antisense therapy are known in the art. (Van der krol, et al., Biotechniques 6:958-976 (1988); Stein, et al., Cancer Res. 48:2659-2668 (1988).

Dosage forms of the inhibitors of BMP-2 and/or BMP-4 of this invention include pharmaceutically acceptable carriers known to those of ordinary skill in the art. Pharmaceutically acceptable components are those that are suitable for use with mammals without undue adverse side effects (such as toxicity, irritation, and allergic response) commensurate with a reasonable benefit/risk ratio. The carrier can be a solid or liquid and the type is generally chosen based on the type of administration being used. The active agent can be coadministered in the form of a tablet or capsule, as an agglomerated powder or in a liquid form. Examples of suitable solid carriers include lactose, sucrose, gelatin and agar. Capsule or tablets can be easily formulated and can be made easy to swallow or chew; other solid forms include granules and bulk powders. Tablets may contain suitable binders, lubricants, diluents, disintegrating agents, coloring agents, flavoring agents, flow-inducing agents and melting agents. Examples of suitable liquid dosage forms include solutions or suspensions in water, pharmaceutically acceptable fats and oils, alcohols or other organic solvents, including esters, emulsions, syrups or elixirs, suspensions, solutions and/ or suspension reconstituted from non-effervescent preparations reconstituted from effervescent granules. Such liquid dosage forms may contain, for example, suitable solvents, preservatives, emulsifying agents, suspending agents, diluents, sweeteners, thickeners and melting agents. Parenteral and intravenous forms may also include isotonic salts and other materials to make them compatible with the type of injection or delivery system chosen.

For administration of an antibody to BMP-2 and/or BMP-4, the pharmaceutically acceptable carrier will usually be an aqueous solution, such as normal saline or phosphate-buffered saline (PBS), Ringer's solution, lactate-Ringer's solution, or any isotonic physiologically acceptable solution for administration by the chosen means. In addition to additives for adjusting pH or tonicity, the antibody may be stabilized against aggregation and polymerization with amino acids and non-ionic detergents, polysorbate, and polyethylene glycol. Optionally, additional stabilizers may include various physiologically-acceptable coarbohydrates and salts. Also, polyvinylpyrrolidone may be added in addition to the amino acid. Suitable therapeutic immunoglobulin solutions, which are stabilized for storage and administration to humans are described in U.S. Pat. No. 5,945,098. Other agents, such as human serum albumin (HAS), may be added to the pharmaceutical composition to stabilize the antibody conjugates.

The method of administration can be any suitable method that effectively alleviates the particular cancer being treated. Possible methods of administration are oral, rectal, parenteral, enterical, subcutaneous, transdermal, peritoneal, intratumoral, or intravenous.

Any suitable dosage of the compounds may be given in the method of the invention. Dosage levels and requirements are well-recognized by those of ordinary skill in the art. As one of ordinary skill in the art will appreciate, an amount constituting an effective amount will vary depending on particular factors. For instance, specific dosage and treatment regimens will depend on facts such as the patient's general health profile, the type of cancer being treated, the severity and course of the patient's disorder, other therapeutics being administered to treat the cancer, and the judgment of the treating physician.

The present invention also provides kits for treating cancer using BMP-2 activity inhibitors. For example, such kits can comprise any one or more of the following materials: packaging material, at least one type of BMP-2 activity inhibitor and/or at least one type of BMP-4 activity inhibitor, and instructions regarding dosage, method of administration, or the like for using the inhibitor to treat cancer.

Detection of BMP-2 to Aid in Diagnosis of Cancer

In addition to its therapeutic aspects, the present invention also relates to a diagnostic method for detecting the presence of elevated levels of BMP-2 and/or BMP-4 in the patient. Applicant has shown that BMP-2 is expressed in many common cancers. In addition, gene expression of BMP-4, a protein that is highly homologous to BMP-2 and has the same biological activity as BMP-2, has been detected in lung cancer tumors. BMP-4 shares 92% homology with BMP-2. Elevated levels of BMP-2 and/or BMP-4 can be detected in various biological samples in mammals, preferably humans. Applicants have shown the presence of BMP-2 in the blood serum of a human patient with cancer. Biological samples, including but not limited to blood, vitreous humor, sputum, aqueous humor, synovial fluid, urine, ascites, and tissue, will be drawn from the patient using standard techniques. Particularly preferred are serum samples.

The measurement of BMP-2 and/or BMP-4 levels may be monitored using any method possible to detect BMP-2 and/or BMP-4 in biological samples. Immunoassays, such as Enzyme Linked Imnmunological Assay (ELISA), Western blots, immunoprecipitation, in situ imunohistochemistry, and immunofluorescence assays are preferred. ELISA is particularly preferred. For a review of general immunoassays, see Stites, D. P., et al., eds., Basic and Clinical Immunology, 8^{th} ed. (Appleton & Lange, Norwalk, Conn.) (1994). Immunological binding assays (or immunoassays) typically use an antibody that specifically binds to a protein or proteins of choice, BMP-2 and/or BMP-4, in this case. The antibody is generally fixed to a substrate such as a plate or a column via covalent or non-covalent linkages (e.g., streptavidin, protein A, protein G, secondary antibodies). Immunoassays also often use a labeling agent to specifically bind to and label the complex formed by the antibody and antigen. The labeling agent may be a labeled anti-BMP-2 antibody and/or a labeled anti-BMP-4 or a labeled antibody that recognizes both BMP-2 and BMP-4. Alternatively, the labeling agent may be a third moiety, such as a secondary antibody, that specifically binds to the antibody/antigen complex.

The immunoassays of this invention may be competitive or noncompetitive. Noncompetitive immunoassays are assays in which the amount of antigen is directly measured. In a “sandwich” assay, for example, the anti-BMP-2 antibodies can be bound directly to a solid substrate on which they are immobilized. These immobilized antibodies then capture BMP-2 in the test sample. BMP-2 thus immobilized is then bound by a labeling agent, such as a second antibody bearing a label. The assay formats may also be performed with anti-BMP-4 antibodies or with antibodies that recognize both BMP-2 and BMP-4. Alternatively, the second antibody may lack a label, but it may, in turn, be bound by a labeled third antibody specific to antibodies of the species from which the second antibody is derived. The second or third antibody is typically modified with a detectable moiety, such as biotin, to which another molecule specifically binds, e.g., streptavidin, to provide a detectable moiety. Methods of binding molecules to a solid support, either covalently or non-covalently, are well known to those of skill in the art. A variety of solid supports known to those of skill in the art, e.g., plate, columns, dipsticks, membranes, and the like, can be used with the present invention.

In competitive assays, the amount of BMP-2 and/or BMP-4 is measured indirectly by measuring the amount of a known modified BMP-2 and/or BMP-4 displaced from a BMP-2 or BMP-4 antibody by the unknown BMP-2 and/or BMP-4 in a sample. In one example of a competitive assay, a known amount of modified BMP-2 is added to a sample and the sample is then contacted with an anti-BMP-2 antibody. The amount of known modified BMP-2 bound to the antibody is inversely proportional to the concentration of BMP-2 in the sample. The amount of modified BMP-2 may be detected by providing a labeled modified BMP-2 molecule.

The label used in the assay is not a critical aspect of the invention, so long as it does not significantly interfere with the specific binding antibody used in the assay. The detectable group can be any material having a detectable physical or chemical property. Thus, a label is any composition detectable by spectroscopic, photochemical, biochemical, immunochemical, electrical, optical, or chemical means. Examples of such labels are magnetic beads, fluorescent dyes, radiolabels, enzymes, and calorimetric labels such as colloidal gold or colored glass or plastic beads.

The label may be coupled directly or indirectly to the desired component of the assay according to methods well known in the art. As indicated above, a wide variety of labels may be used, with the choice of label depending on sensitivity required, ease of conjugation with the compound, stability requirements, available instrumentation, and disposal provisions. Non-radioactive labels are often attached by indirect means. Generally, a ligand molecule, such as biotin, is covalently bound to the molecule. The ligand then binds to another molecule, such as streptavidin, which is either inherently detectable or covalently bound to a signal system, such as a detectable enzyme, a fluorescent compound, or a chemiluminescent compound. The ligands and their targets can be used in any suitable combination with antibodies that recognize BMP-2 and/or BMP-4. The molecules can also be conjugated directly to a signal generating compound, e.g., by conjugation with an enzyme or fluorophore.

Means of detecting labels are well known to those of skill in the art. Thus, for example, where the label is a radioactive label, means for detection include a scintillation counter or photographic film as in autoradiography. Where the label is a fluorescent label, it may be detected by exciting the fluorochrome with the appropriate wavelength of light and detecting the resulting fluorescence. The fluorescence may be detected visually, by means of photographic film, by the use of electronic detectors such as charge coupled devices (CCDs) or photomultipliers or the like. Similarly, enzymatic labels may be detected by providing the appropriate substrates for the enzyme and detecting the resulting reaction product. Finally, simple colorimetric labels may be detected simply by observing the color associated with the label.

Some assay formats do not require the use of labeled components. For instance, agglutination assays can be used to detect the presence of the target antibodies.

Results

Experimental results supporting the above uses of BMP-2 and/or BMP-4 and their inhibitors are set forth in detail below. All of the experimental methods mentioned in this section, such as representational difference analysis, Western blot assays, and immunohistochemical studies, are described in detail in the Examples section that follows.

Identification of BMP-2 Using RDA Subtraction Technique:

Initially, Applicant performed representational difference analysis (RDA) on cDNA derived from normal and cancerous lung tissue samples to identify genes that were uniquely or highly expressed in human lung cancer in comparison to normal tissue. RDA has been described in the literature and allows detection of differences in gene expression between two similar populations. It involves exposing digested tester cDNA ligated to a primer to high concentrations of similarly digested but non-primer bearing driver cDNA, melting the tester and driver cDNA, and allowing them to hybridize. Subsequent PCR results in exponential amplification of the target cDNA of the tester that hybridizes to other tester cDNA. (Hubank, M., Nucleic Acids Research 22:5640-5648 (1994)) Here, Applicant used a non-small cell lung carcinoma (NSCLC) as the tester and immortalized human bronchial epithelial (IHBE) cells as the driver. IHBE cells rather than normal lung tissue were used, as IHBE cells proliferate at a rate that is more similar to human lung carcinomas than to normal lung tissue. Thus, Applicant avoided identifying genes involved in the proliferation cascade but that were not by themselves transforming.

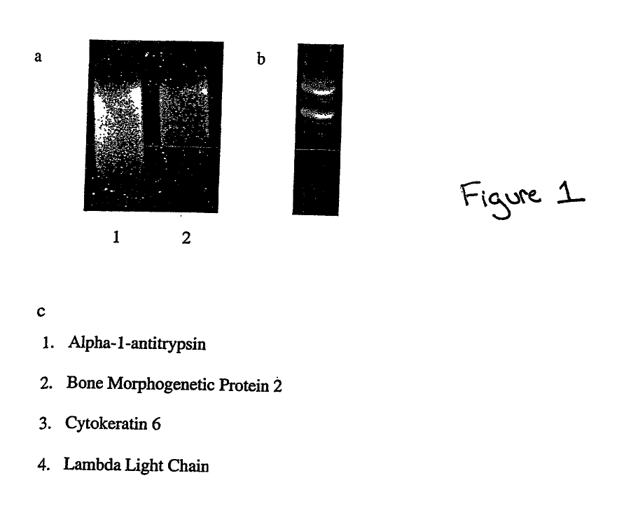

After two rounds of subtraction, several distinct bands, which were cloned and sequenced, were present in the amplified tester cDNA. (FIG. 1b) A BLAST data base search identified BMP-2 expression in the lung tumor tissue specimen as well as expression of alpha-1-antitrypsin, cytokeratin 6, and lambda light. (FIG. 1c)

Expression of BMP-2 In Various Cancer Tissue Specimens, Cancer Cell Lines, and Blood Serum from a Cancer Patient

Using reverse transcriptase polymerase chain reaction (RT-PCR), Western blots, and immunohistochemical assays to study the expression of BMP-2, BMP-4 and their receptors in various tissue specimens and in cell lines, Applicant found that BMP-2 was highly expressed in many types of cancers. Applicant also detected gene expression of BMP-4 in human lung cancer tumor samples.

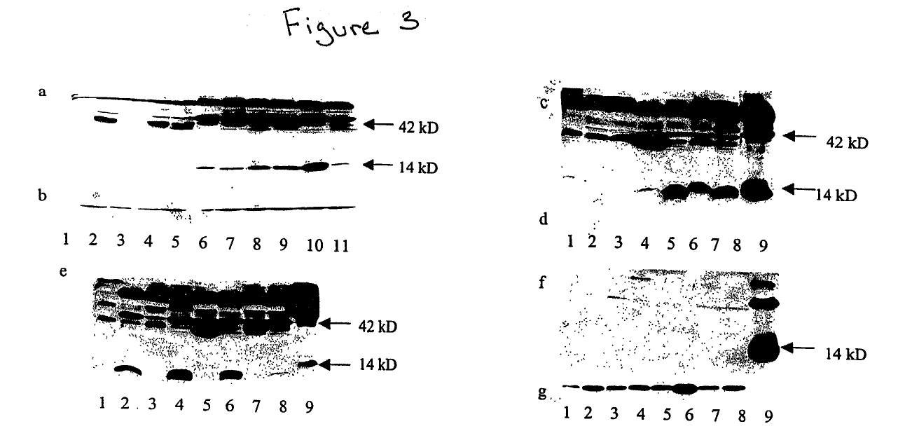

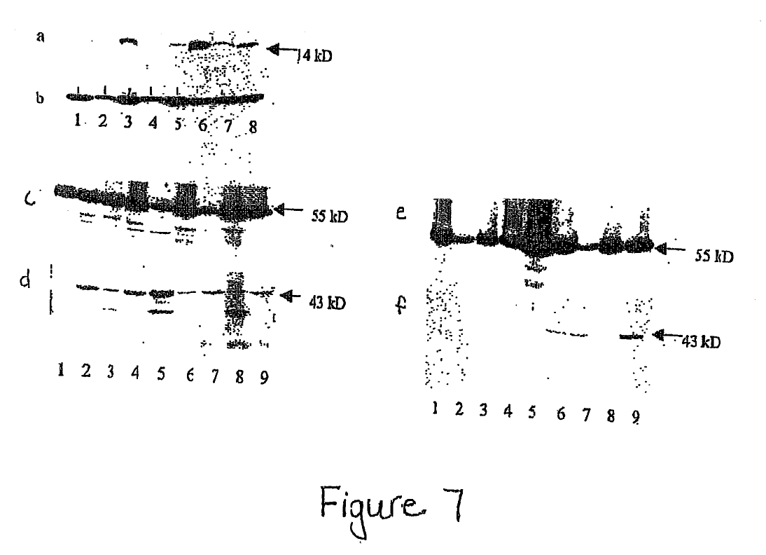

Applicant performed his initial experiments on normal and cancerous lung tissue and lung cancer cell lines. Using Western blot analysis, Applicant found that the mature active 14 kD BMP-2 protein was aberrantly expressed in almost all of the 25 non-small cell lung carcinoma (NSCLC) tissue specimens examined There was little to no expression of BMP-2 in 11 normal lung tissue specimens. A representative Western blot is shown in FIG. 3. An anti-actin immunoblot showed near equal loading of the samples. (FIG. 3(b)) In addition, BMP-2 was found to be highly expressed in all epithelial derived lung carcinomas of which NSCLC is derived and in the rare malignant neuroendocrine tumor. (FIG. 3(c) and FIG. 3(e), Lane 4, respectively) Western blot analysis of each of the different cell types comprising NSCLC—adeno, squamous, large cell, and bronchoalveolar carcinomas—revealed that the level of BMP-2 expression was not dependent on the cell type or whether the tumor was well or poorly differentiated. In comparison, the level of BMP-2 expression in benign lung tumors (FIG. 3(e), Lane 1) and inflammatory diseases of the lung (FIG. 7(a), Lane 1) was very low, similar to that seen in normal lung tissue, showing that BMP-2 is not an acute phase protein and that high levels of BMP-2 expression are indicative of malignant tumors. Neither BMP-4 nor BMP-7 expression was detected in the lung tissue specimens or the A549, H7249, IHBE, and NBE cell lines by Western analysis. (FIG. 3(f)) But BMP-4 gene expression was detected in RT-PCR experiments on cancerous human lung tissue specimens. And BMP-4 expression was detected by Western blot analysis in the tumors that developed in nude mice injected with A549 cells transfected with expression vectors encoding either BMP inhibitors or BMP receptor inhibitors. The results of both of these experiments will be discussed in more detail below.

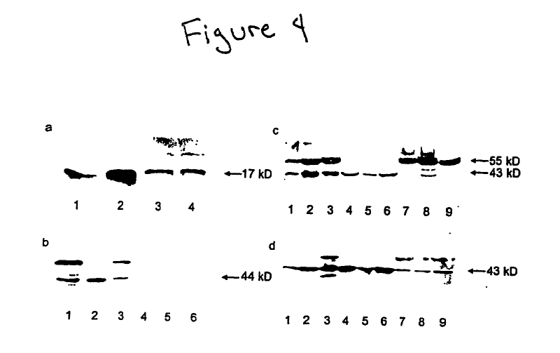

Applicant also tested for expression of BMP-2 in various lung cancer and normal cell lines. Although the mature BMP-2 protein was detected in the cell lysate of the A549 and H7249 human lung cancer cell lines, the level of expression was not significantly different from the level of expression in the cell lysate of immortalized normal human bronchial epithelial cells (IHBE). (FIG. 4(a)) Because BMP-2 is a secreted protein, Applicant also examined its expression in the cell culture media. A Western blot of the cell culture media showed the A549 and H7249 cell lines secreted a 43 kD BMP-2 precursor protein. (FIG. 4(b), Lanes 2-3) This BMP-2 precursor was not detected in the media from either the IHBE or normal bronchial epithelial (NBE) cells (FIG. 4(b), Lanes 4-5).

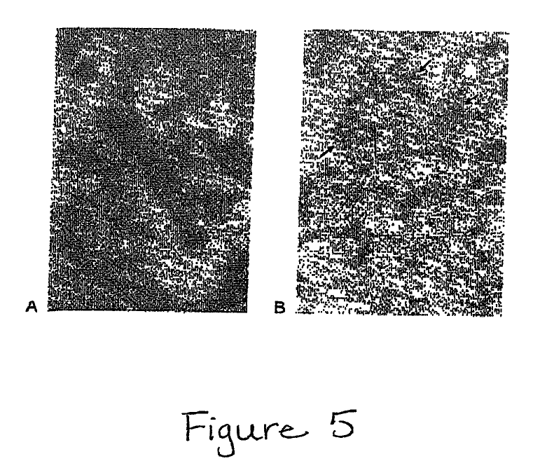

Immunohistochemistry studies of patient derived NSCLC also localized the expression of BMP-2 to the cancer cells (FIG. 5(a)). Absorbing the anti-BMP-2 antibody with recombinant human BMP-2 completely inhibited staining of the tumors (FIG. 5(b)). BMP-2 expression was not detected in normal lung tissue by immunohistochemistry.

Applicant turned next to receptors and found that normal and cancer lung tissue specimens and cell lines express both type IA and IB BMP receptors. The lung cancer and normal lung tissue specimens express a 55 kD and 44 kD type IA BMP-2 receptor. The tumor specimens expressed predominately the 55 kD receptor, while normal lung tissue specimens expressed a higher percentage of the 44 kD receptor. The A549, H7249, and IHBE cells only expressed a 44 kD type IA BMP receptor. (FIG. 4(c)) The tissue specimens and cell lines expressed a 44 kD type IB BMP receptor with normal lung tissue demonstrating more expression than that of the tumor specimens. (FIG. 4(d))

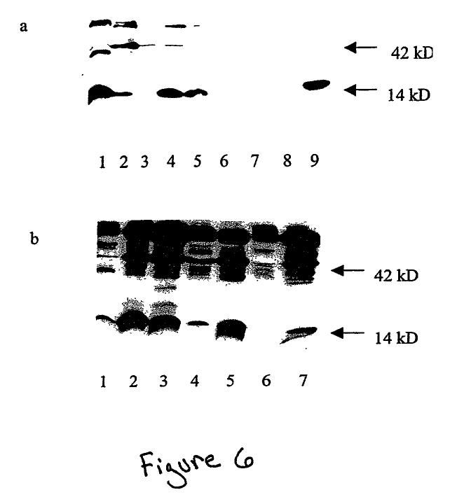

Similar to their findings with lung tissue, Applicants found that BMP-2 was expressed in many other common human malignancies but not in their corresponding normal tissues. Western blot analysis revealed that BMP-2 was overexpressed in breast, bladder, colon, endometrial, omental, and kidney carcinomas with low levels of BMP-2 expression in the corresponding normal tissue. (FIGS. 6(a) and (b)). BMP-2 was also found to be expressed in ovarian (FIG. 6(b), lane 3), mesothelioma (FIG. 3(e), lane 2), thyroid, hepatoma, and testicular carcinoma.

BMP-2 and its receptors were also examined in both primary and metastatic carcinomas that were surgically removed from patients. BMP-2 was found to be highly expressed in kidney tumors that had metastasized to the lung, a metastatic breast cancer to chest wall cavity, and a NSCLC lung tumor that had metastasized to a regional lymph node. (FIG. 7(a)) The BMP IA receptor was expressed equally between the primary and metastatic carcinomas and the corresponding normal tissue (FIG. 7). The BMP IB receptor was expressed in all metastatic and primary tumors examined. (FIG. 7) The BMP IB receptor, in contrast to the BMP IA receptor, was not expressed in all the corresponding normal tissues. While it was expressed in normal lung tissue with slight expression in normal endometrium it was not expressed in normal kidney, colon, and omentum. (FIG. 7(f)) Interestingly, the IB receptor was expressed in both primary and metastatic renal carcinoma, but not in normal kidney tissue. (FIG. 7(f), Lane 6)

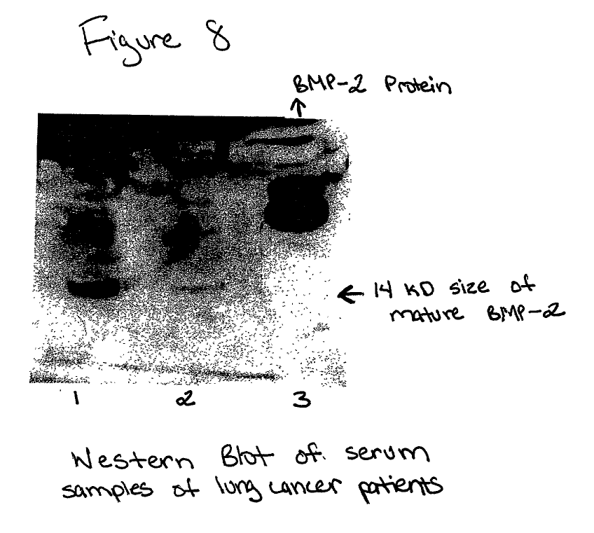

BMP-2 expression was also found in blood serum samples from lung cancer patients. (FIG. 8)

Processing of Inactive BMP-2 Precursors

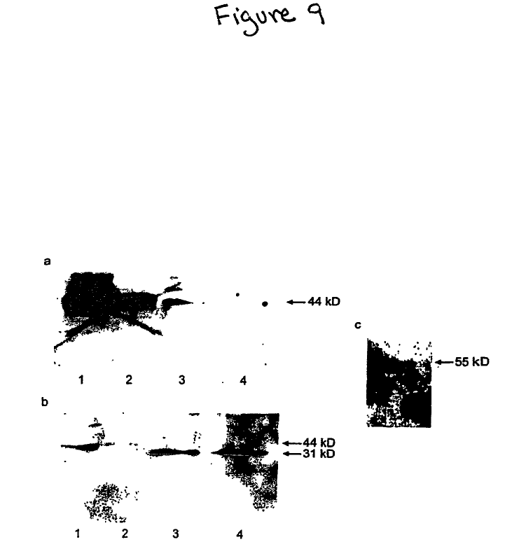

Because BMP precursors are proteolytically activated by proprotein convertases, Applicant studied whether BMP-2 could be processed following secretion, hypothesizing that secreted BMP-2 precursors from tumor cells may be processed by cells present in the tumor stroma. Because leukocytes normally infiltrate lung and furin convertase is ubiquitously expressed, the ability of leukocytes to cleave proprotein BMP-2 secreted from A549 cells was examined. First, Applicant determined that the furin convertase is expressed in human leukocytes isolated from whole blood. (FIG. 9(c)). Human leukocytes were incubated with A549 cell culture media containing the BMP-2 precursor protein. A Western blot of the incubated media samples was probed with an anti-human BMP-2 precursor antibody that recognizes its C-terminal end. The 45 kD BMP-2 precursor protein was consistently decreased following incubation with the leukocytes (FIG. 9(a)). By probing immunoblots with an anti-human BMP-2 antibody that recognizes its N-terminal end, Applicant identified a 31 kD BMP-2 product present only in the media samples incubated with leukocytes. (FIG. 9(b)) This data shows that BMP-2 precursor proteins are cleaved by human leukocytes.

Effect of BMP-2 on Tumors and Cancer Cell Lines

After determining that BMP-2 was highly expressed in most common cancers, Applicant performed experiments to show that BMP-2 causes cancer invasion and metastasis. Applicant performed experiments with lung cancer cell lines and with nude mice injected with A549 cells.

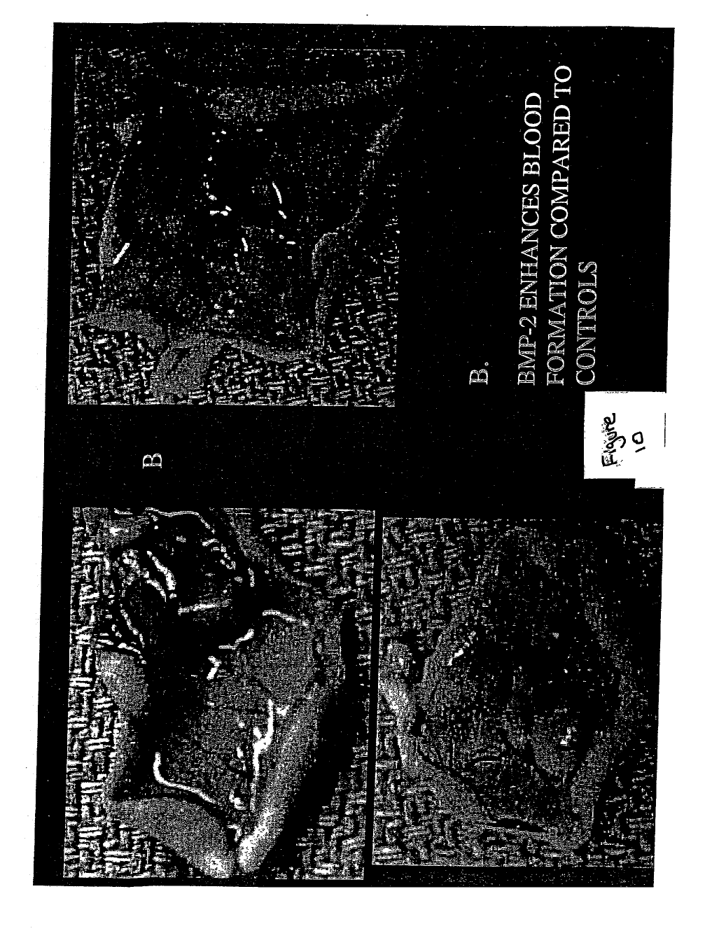

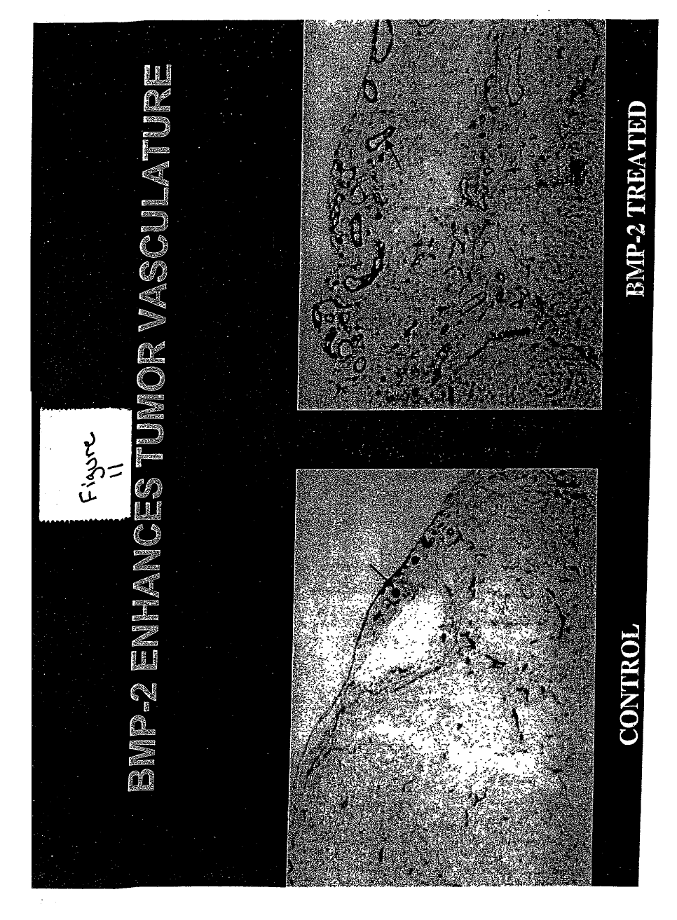

The experiments with the nude mice showed that BMP-2 treatment enhances blood vessel formation around tumors from nude mice injected with A549 cells. Some of the mice were co-injected with BMP-2. Gross observations of tissue harvested after six days showed that the addition of recombinant BMP-2 to developing tumors in nude mice caused increased blood vessel formation. (FIG. 10) Tissue was also stained with anti-CD 31 antibody which recognizes endothelial cells. A person blind to how the tumors were created then observed the tissue through a microscope and counted the number of vessels that had formed in the tumor. This data showed that BMP-2 caused a statistically significant increase in the number of blood vessels in the tumor. (FIG. 11)

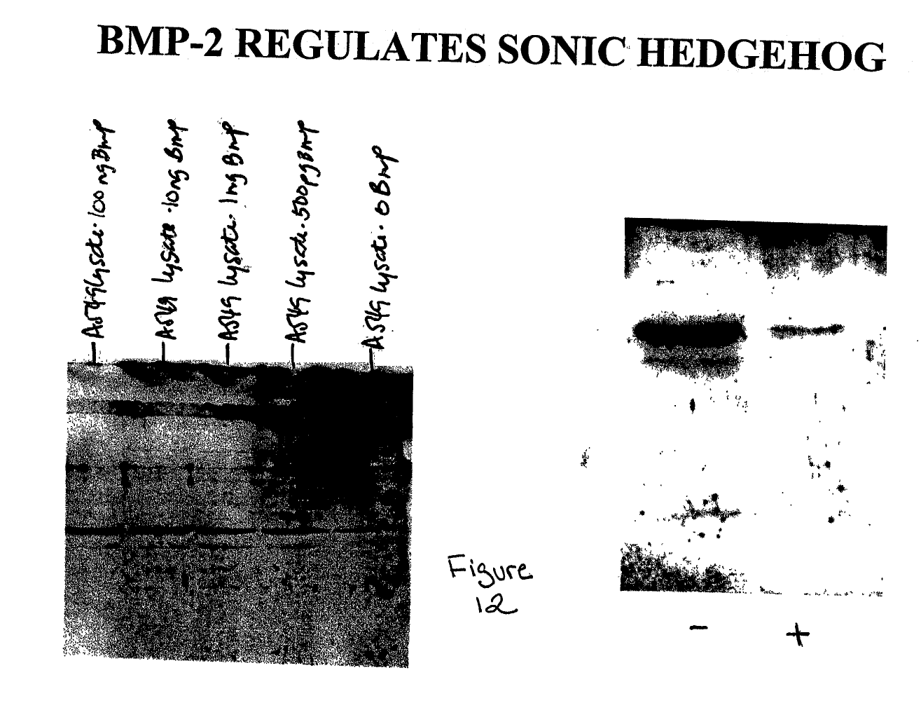

Other studies showed that addition of BMP-2 to cancer cell lines increased expression of vascular endothelial growth factor (VEGF) and the oncogene Sonic Hedgehog. VEGF is the most potent angiogenic factor and is though to be essential for tumor growth and metastasis. (Folkman, J. J. Nat'l Cancer Inst. 82:4 (1990); Zetter, B. Annual Rev. Med. 49:407 (1998); Ferrara, N. Current Topics Microbiol. Immunol. 237:1 (1999)) Transgenic mice studies have confirmed that overexpression of sonic hedgehog can cause tissue-targeted cancer. (Oro, A. E., et al., “Basal carcinomas in mice overexpressing sonic hedgehog” Science 276: 817-21 (1997)) The addition of recombinant BMP-2 to human aortic endothelial cells in culture caused an increase in VEGF secretion as determined by ELISA performed on the cell culture media. The concentration of VEGF in the cell culture media before treatment with BMP-2 was 11.2 pg/ml. The VEGF concentration after treatment with 0.500 pg/ml BMP-2 was 233.0 pg/ml and after treatment with 1 ng/ml BMP-2 was 2,969.0 pg/ml. The addition of increasing amounts of BMP-2 to lung A549 lung cancer cells growing in culture also caused a dose responsive increase in the expression of the oncogene Sonic Hedgehog. (FIG. 12)

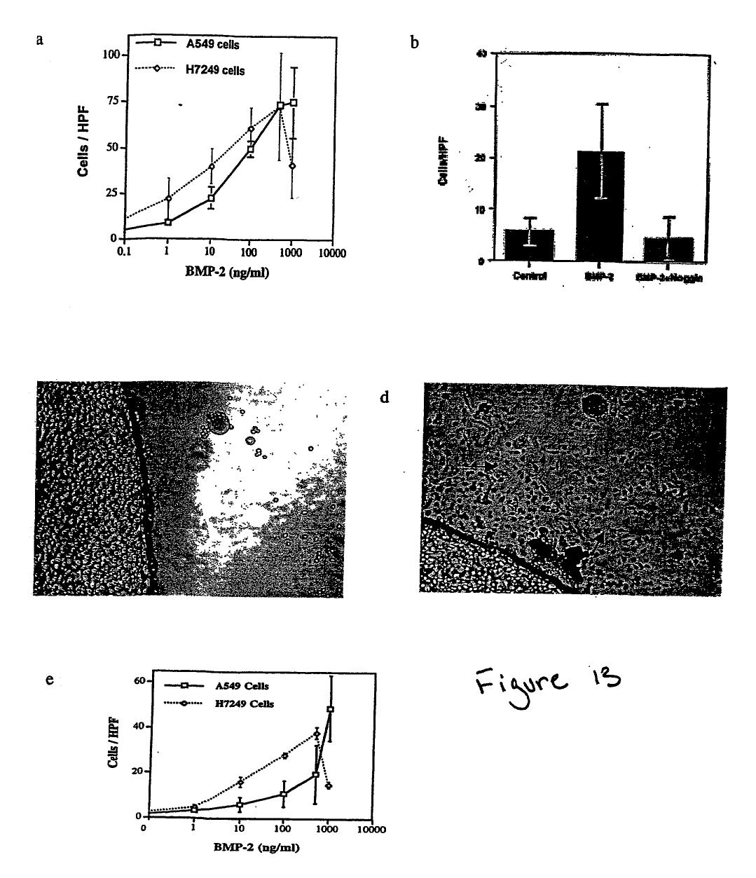

In addition, Applicants showed that BMP-2 stimulates the migration and invasion of the human lung cancer cell lines A549 and H7249. In one assay, recombinant BMP-2 caused a dose responsive increase in migration of cells from transwell migration chambers. (FIG. 13(a)) In another, BMP-2 stimulated the migration of A549 and H7249 cells cultured on glass cover slips toward Affi-blue agarose beads containing recombinant BMP-2. (FIG. 13(c) and (d)) In addition, using transwell chambers coated with Matrigel, Applicants also showed that recombinant BMP-2 caused a dose responsive increase in the invasion of both A549 and H7249 cells. (FIG. 13(e))

Effects of Inhibiting BMP-2 Expression

After finding that BMP-2 enhances cancer invasion and growth, Applicant conducted experiments to determine whether inhibitors of the activity of BMP-2, including anti-BMP-2 antibodies, could be used to treat cancer. In these studies, recombinant mouse noggin (R & D Systems, Minneapolis, Minn.) was used as a representative inhibitor. Noggin, a natural inhibitor of BMP-2, is a secreted protein that binds BMP-2 and BMP-4, thereby preventing their activation of the BMP receptors. (Weaver, M., et al., Development 126: 4005-4115 (1999); Zimmerman, L. B., et al., Cell 86: 599-606 (1996); Tucker, A.S., et al., Science 282: 1136-1138 (1998); Capdevilla, J., et al., Developmental Biology 197: 205-217 (1998); Brunet, L. J., et al., Science 280: 1455-1447 (1998)) Mouse and human noggin are 98% homologous.

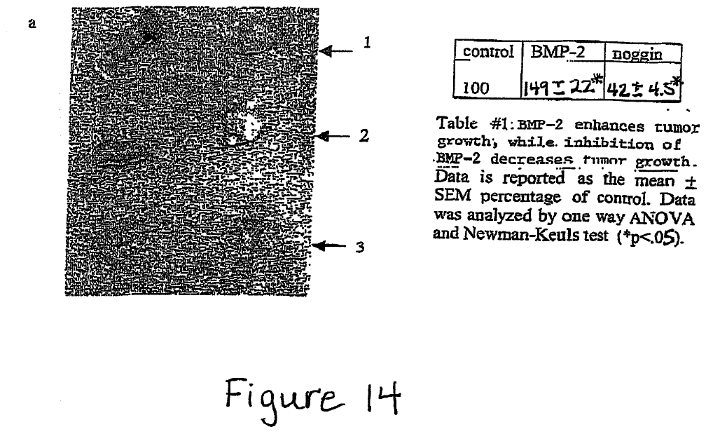

The effects of BMP-2 and noggin on tumor growth in vivo was examined by co-injecting the A549 cells subcutaneously into nude mice with Affi-Blue agarose beads coated with either albumin, recombinant human BMP-2, or recombinant human noggin. The animals were then sacrificed and tumors measured at 12 or 19 days. Inhibiting BMP-2 activity with noggin resulted in a statistically significant decrease in tumor growth. Addition of BMP-2 resulted in a statistically significant increase in tumor growth. (FIG. 14)

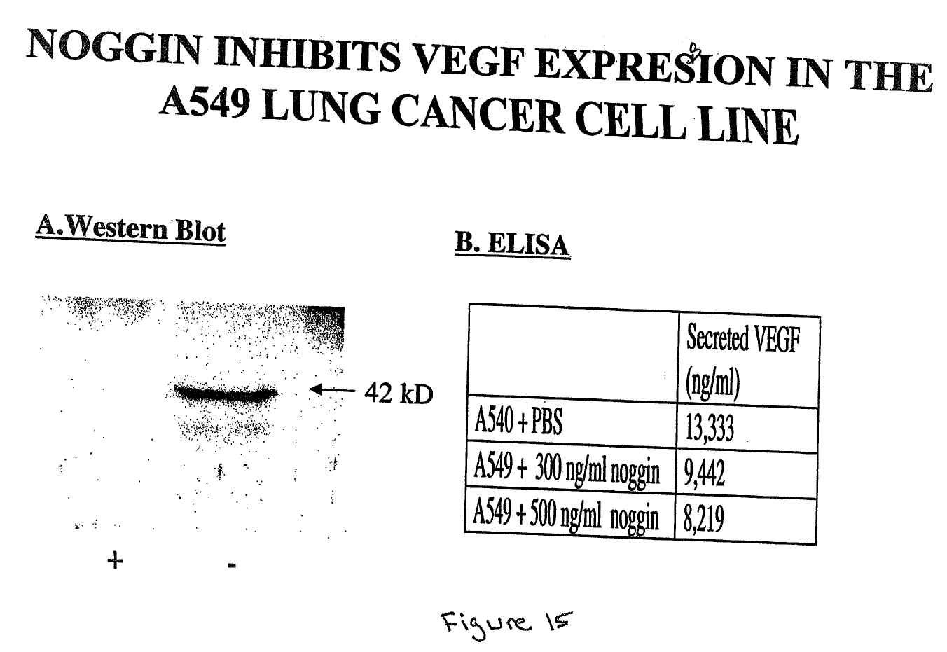

When added to A549 cells noggin decreased the expression of VEGF and sonic hedgehog (FIGS. 12 and 15). Noggin also decreased proliferation of A549 cells growing in culture.

Applicant also found that noggin completely inhibited the ability of BMP-2, discussed above, to enhance the migration of the A549 cells in a transwell chamber. (FIG. 13(b))

Applicant also studied the effects of an anti-BMP-2 antibody on tumor growth in vivo. This was examined by co-injecting the A549 cells subcutaneously into nude mice with either the anti-BMP-2 antibody or with an isotype control antibody. The animals were sacrificed and tumors harvested after three weeks. The addition of the anti-BMP-2 antibody resulted in a statistically significant decrease in tumor growth.

Gene Expression of BMP-4 in Human Lung Cancer Tumor Specimens

In addition to his findings regarding BMP-2, Applicant detected BMP-4 expression in human lung cancer tumor samples. Sequencing of cDNA obtained from RT-PCR performed on human lung cancer tumors revealed the expression of BMP-4 in nine out of ten samples examined. As is discussed above, BMP-4 is highly homologous to BMP-2; it is inhibited by the same inhibitors that antagonize BMP-2 and binds to and activates the same receptors that BMP-2 activates.

Expression of BMP-4 in Nude Mice

Using Western blot analysis, Applicant also found expression of BMP-4 in the tumors of nude mice that had been injected either with 1) A549 cells transfected with expression vectors containing noggin, which inhibits both BMP-2 and BMP-4, 2) A549 cells transfected with expression vectors containing BMP receptor antagonists or 3) A549 cells transfected with expression vectors containing green fluorescent protein (GFP). Applicant compared the signals on the Western blot corresponding to the tumors from mice injected with the transfected cells to the signal for the control-recombinant BMP-4. There was a strong signal for the tumors resulting from the cells transfected with the noggin expression vector and a fair signal for the tumors resulting from the cells transfected with the GFP expression vector.

### EXAMPLES

### Example 1

Identification of BMP-2 Using Representational Difference Analysis (RDA) Subtraction Technique

Representational difference analysis (RDA) subtraction technique was used to identify genes highly expressed in a non-small cell lung carcinoma obtained from a patient (tester) in comparison to normal bronchial human epithelial cells (driver). The technique for RDA described in the following references was followed: Holmes, M. L., et al., Molecular and Cellular Biology 19: 4182-4190 (1999); Hubank, M., Nucleic Acids Research 22:5640-5648 (1994). Normal human bronchial epithelial cells were purchased from Clonetics, BioWhitaker (Walkersville, Md.) and were maintained in serum free media. Human tissue specimens were obtained directly from the operating room and immediately frozen in liquid nitrogen. Tissue was stored in liquid nitrogen at −70 C.

To perform RDA, MRNA was purified from the samples using Oligo dT columns (Pharnacia, Peapack, N.J.) according to the manufacturer's instructions and cDNA was then obtained using the Pharmacia Time Saver cDNA synthesis kit also according to the manufacturer's instructions. cDNA was digested with Sau3A I endonuclease, R-linker ligated, and amplified by PCR. The R-linkers were removed and J-linkers ligated to the tester. The driver and tester cDNA were hybridized at 67 C. for 20 hours (driver in excess 100:1) and the subtracted tester cDNA amplified by PCR. A second round of subtraction was performed using N-linkers (driver in excess 800,000: 1). The amplified PCR products were cloned into blue script and sequenced using a EBI Prism 377 DNA sequencer. Known genes corresponding to the subtracted tumor cDNA were identified by a BLAST database search.

### Example 2

Detection of Over-Expression of BMP and BMP Receptors in Various Cancer Tissue Specimens and Lung Cancer Cell Lines

Applicant detected expression of BMP and BMP receptors in a number of normal and cancerous tissue specimens and cells. As described above, all human tissue specimens were obtained directly from the operating room and immediately frozen in liquid nitrogen and stored at −70° C. Normal human bronchial epithelial (NBE) cells were purchased from Clonetics, BioWhitaker (Walkersville, Md.) and were maintained in serum free media. Immortalized human bronchial epithelial (IHBE), BEAS-2B, cells were derived from normal bronchial epithelial cells immortalized with an adenovirus-12-5V40 hybrid virus (32). A549 and H7249 are highly invasive human lung cancer cell lines. The cell lines were cultured in 5% fetal bovine serum (FBS) in Dulbecco's Modified Eagles medium (DME) containing penicillin, streptomycin, and glutamine with 5% pCO2 at 37° C. Western blot analysis was used to detect expression of the BMP ligand and its receptors in all of these samples. Immunohistochemistry studies were performed to detect BMP in non-small cell lung carcinoma samples and normal lung tissue samples derived from patients.

Western Blot Analysis

In preparation for Western blot analysis, cells were lysed in a modified RIPA buffer containing 150 ml NaCl, 50 ml tris, pH 7.5, 1% NP 40, 10% deoxycholic acid, and protease inhibitor cocktail from Calbiochem. Tissue specimens were sonicated on ice in the same modified RIPA buffer. The protein concentration of the resulting samples was measured using the Bradford assay technique. Recombinant human BMP-2, purchased from R & D Systems and reconstituted in PBS with gelatin, served as a control. Total cellular protein of the samples and recombinant human BMP-2 were analyzed by SDS-PAGE, transferred to nitrocellulose filter (Schleicher and Schuell, Keene, N.H.) at 35 V for 16 hours at 4° C. and then incubated with the desired primary antibody. Specific proteins were detected using the enhanced chemiluminescence system (Amersham, Arlington Heights, Ill.).

The primary antibodies that were used included mouse anti-human BMP-2, goat anti-human BMP-4, goat anti-human BMP-7, goat anti-human type IA BMP receptor, and goat anti-human type IB BMP-2 receptor. All of these antibodies, except the goat anti-human BMP-7 were purchased from R & D Systems in Minneapolis, Minn. The goat anti-human BMP-7 antibody were obtained from Santa Cruz (Santa Cruz, Calif.).

Immunohistochemistry Analysis

To perform immunohistochemistry analysis, four micron Cryostat-cut sections were air dried before being fixed in cold acetone for 10 minutes. Sections were washed in cold 0.5 M PBS and intrinsic peroxidase was quenched with 0.03% periodic acid for 20 minutes at room temperature. Sections were then rinsed in cold PBS and 0.5% BSA/PBS was applied to the slides for 15 minutes in a humid chamber. Biotinylated BMP-2/4 (R & D Systems) was applied at a 1:25 dilution in 1% BSA/PBS and incubated overnight at 4 C. Two slides were run as negative controls. One slide was incubated with biotinylated BMP-2 preabsorbed with recombinant human BMP-2 at 1:10 Molar ratio. As a second negative control slide samples were incubated overnight at 4 C. with normal rabbit serum. Slides were washed with cold PBS and incubated for one hour in Streptavidin horseradish peroxidase (Dako) at a 1:500 dilution in 1% BSA/PBS. Slides were then counterstained in 0.7% Toluidine Blue.

### Example 3

Detection of Processing of Mature BMP-2 by Human Leukocytes

Cell culture media from the A549 cells was incubated with leukocytes isolated from whole blood for 16 hours. Then, a Western blot was performed, as described above, on the cell culture media. Mouse anti-human BMP-2 antibody (#MAB355, R & D Systems, Minneapolis, Minn.) was the primary antibody used to detect the C-terminal end of BMP-2. Goat anti-human BMP-2 (Research Diagnostics, Flanders, N.J.) was used to detect the N-terminal end of BMP-2. A Western blot of the leukocytes was also performed with an anti-furin primary antibody to determine that human leukocytes express furin convertase.

### Example 4

Analysis of the Effect of BMP-2 and Noggin on Tumor Growth and Tumor Vasculature In Vivo

Nude mice studies were conducted to determine the effect of BMP-2 and one of its inhibitors, noggin, on tumor growth and tumor vasculature. 10⁶ A549 cells were injected subcutaneously into nude mice with Affi-Blue agarose beads coated with albumin, recombinant human BMP-2 or recombinant mouse noggin. Both of these recombinant proteins were purchased from R & D Systems and were reconstituted in PBS with gelatin. Coating of Affi-Blue agarose beads with BMP-2 and noggin has been described in the literature. (Abe, E., et al., J. Bone Miner Res. 15: 663-673 (2000); Tucker, A. S., et al., Science 282: 1136-1138 (1998); Zimmerman, L. B., et al., Cell 86: 599-606 (1996)) In brief, 25 ug of Affi-blue agarose beads were incubated with 100 ug/ml albumin, recombinant human BMP-2, or recombinant noggin for 2 hours and then washed 3 times with PBS immediately prior to use. In separate experiments the beads were not washed prior to injection. The coated beads were injected with the A549 cells into nude mice subcutaneously. To assess tumor growth after 12 or 19 days the length, width, and depth of the tumors were measured in mm. To assess tumor vasculature, tissue including a tumor was harvested after six days. Gross observations of the tissue were made. In addition, the tissue was stained with anti-CD 31 antibody, which recognizes endothelial cells. Vessels in five high power fields were counted by a person blinded to how the tumors were created.

Nude mice studies were also conducted to determine the effect of an anti-BMP-2 antibody on tumor growth. As with the experiments with noggin and BMP-2 described above, 10⁶ A549 cells were co-injected subcutaneously into nude mice with either the anti-BMP-2 antibody (Genetics Institute, Andover, Mass.) or with an isotype control antibody. Tumors were harvested after three weeks and tumor growth assessed by measuring the length, width, and depth of the tumors in mm.

### Example 5

Effect of BMP-2 and Noggin on VEGF and Sonic Hedgehog Expression

Western blot analvsis of VEGF and sonic hedgehog in presence of BMP-2 and noggin

Western blots, as described above, were performed on total cellular protein samples and cell culture media samples. The primary antibodies used to detect VEGF and sonic hedgehog were anti human VEGF from R & D Systems (Minneapolis, Minn.) and anti human sonic hedgehog from Santa Cruz (Santa Cruz, Calif.), respectively.

ELISA of VEGF in presence of BMP-2 and Various Concentrations of Noggin

The sandwich ELISA method was used to determine VEGF concentrations in the cell culture media of A549 cells treated with noggin and in the cell culture media of human aortic endothelial cells treated with BMP-2. 100 ul of the monoclonal capture antibody, diluted in carbonate buffer (sodium bicarbonate, sodium carbonate, pH 9.0), was added to each well of a MaxiSorb Nunc-immuno plate and incubated overnight at 4 C. The plates were washed two times with washing buffer (1× PBS with 0.0005% tween-20). Then, 200 ul of blocking buffer (1× PBS with 1% BSA) was added per well and incubated for 2 hours at room temperature. The plates were then washed 4 times with washing buffer.

The recombinant protein standards and samples (100 ul per well) were added and the plate was then incubated overnight at 4 C. The plates were washed 5 times with washing buffer. The biotinylated detection antibody was diluted in incubation buffer (1× PBS with 10% fetal bovine serum) for a final concentration of 1 ug/ml. 100 ul of the detection antibody was added per well and incubated for 1 hour on a shaker at room temperature. The plates were washed 6 times with washing buffer and 100 ul of streptavidin-HRP conjugate (1:10,000) was added per well. The plates were incubated for 45 minutes at room temperature on a shaker and then washed 6 times with washing buffer. 100 ul/well of the substrate reagent (0.2 M citrate buffer, 1 mg/ml o-phenylenediamine dihydrocholoride (OPG), 3% hydrogen peroxide) was added and covered with aluminum foil for ten minutes. The reaction was stopped with 100 ul/well of 2M sulfuiric acid and absorbance determined using an automated plate reader with a 490/690 filter. The protein concentration was then determined from the standard curve.

### Example 6

Analysis of Effect of Noggin on A549 Cell Growth

A549 cell cultures grown in DME media with fetal calf serum were treated with recombinant mouse noggin. (R & D Systems) Using a hemacytometer, cell counts were then taken after two days and after four days. Growth suppression was seen at 1 ng/ml noggin.

Example 7

Identification of BMP-2 as a Stimulant of Human Lung Cancer Cell Migration and Invasion

Migration Assay In Monolayer Culture

To detect BMP-induced migration in a monolayer culture, recombinant human BMP-2 (R & D systems, Minneapolis, Minn.) was coated to Affi-Blue agarose beads (Bio Rad, Hercules, Calif.) as described in the literature. (Vainio, S.; et al., Cell 75: 45-58 (1993); Sloan, A. J., et al., Arch Oral Biol. 44: 149-156 (1999)) Briefly, 100 ml of the Affi-Blue agarose beads were incubated with either 10 ml of recombinant BMP2 reconstituted in PBS with gelatin (100 mg/ml) or PBS alone at 37° C. for 2 hours, washed with PBS, and reconstituted with 40 ml of PBS. Glass cover slips were coated with serum free media containing BSA, fibronectin and collagen (32) and 50,000 cells were plated per cover slip in serum free media. Two microliters of the Affi-Blue agarose beads coated with recombinant BMP-2 or dilution buffer were placed in linear fashion next to the cover slips.

Chemotactic Assay

In the chemotactic assay, fifty thousand cells were placed in the upper chamber of an 8 micron transwell migration chamber (Becton Dickinson, Bedford, Mass.) and 300 ml of serum free media with 0 ng/ml, 1 ng/ml, 10 ng/ml, 100 ng/ml, 500 ng/ml, or 1000 ng/ml recombinant human BMP-2 placed in the lower well. After 24 hours the filters were then removed and the top of the filter wiped with a cotton swab and the cells that migrated through the filters were stained with Syto-16 intercalating dye. Five high power fields were counted using fluorescent microscopy. To show that noggin inhibits BMP-2 induced migration, the experiment was also performed with each of the following in the lower well of the transwell chamber: media alone, recombinant BMP-2 (500 ng/ml), and noggin (10 ug/ml) with recombinant BMP-2 (500 ng/ml).

Matrigel Invasion Assay

Invasion was studied using transwell chambers coated with 100 ml of Matrigel (Becton Dickinson). Fifty thousand cells were placed in the upper chamber and 300 ml of serum free media with 0 ng/ml, 10 ng/ml, 100 ng/ml, 500 ng/ml, or 1000 ng/ml recombinant BMP-2 placed in the lower wells. After 48 hours the Matrigel was removed and cells that had migrated through the filter were stained with Syto-16 intercalating dye and 5 high power fields counted using fluorescent microscopy.

### Example 8

Detection of Gene Expression of BMP-4 in Human Lung Cancer Specimens Using RT-PCR and Sequencing

Reverse transcriptase polymerase chain reaction was performed using standard techniques well known in the art. The forward primer was acgagagctctcactggtcc (SEQ ID No: 15). The reverse primer was cattccggattacatgaggg (SEQ ID No: 16). The chain reaction consisted of denaturation at 95 C. for 1 min, annealing at 54 C. for 1 min, and extension at 72 C. for 2 min with 33 cycles. The resulting cDNA was sequenced at a core facility at the University of Medicine and Dentistry of New Jersey, using an automated sequencer.

### Example 9

Detection of Expression of BMP-4 in Tumors of Nude Mice Injected with A549 Cells Transfected with Various Constructs

A549 cells were transfected with expression vectors that express green fluorescent protein (GFP), bone morphogenetic protein receptor IA antagonist, bone morphogenetic protein receptor IB antagonist, and noggin. 10⁶ of each of the transfected cells were then injected subcutaneously into nude mice. The resulting tumors were harvested after three weeks. Western blots, as described above, were performed on total cellular protein samples. The primary antibody used was goat anti-human BMP-4 and was purchased from R & D Systems. The recombinant BMP-4 used as a control was a human recombinant and was also purchased from R & D Systems.

1. A method for the treatment of cancer comprising administering to a patient a therapeutically effective amount of a bone morphogenetic protein-4 activity inhibitor.

2. The method of claim 1 wherein the bone morphogenetic protein-4 activity inhibitor is a polypeptide that binds specifically to bone morphogenetic protein-4.

3. The method of claim 1 wherein the bone morphogenetic protein-4 activity inhibitor is a polypeptide that binds specifically to a bone morphogenetic protein-4 receptor.

4. The method of claim 3 wherein the bone morphogenetic protein-4 receptor is a bone imorphogenetic protein IB receptor.

5. The method of claim 1 wherein the bone morphogenetic protein-4 activity inhibitor is selected from the group consisting of noggin, chordin, cerberus 1 homolog, and gremlin.

6. The method of claim 1 wherein the bone morphogenetic protein-4 activity inhibitor is noggin.

7. The method of claim 6 wherein the amino acid sequence of noggin is selected from the group consisting of amino acids #20-231 of SEQ ID NO: 4 and amino acids #20-231 of SEQ ID No: 6.

8. The method of claim 1 wherein the bone morphogenetic protein-4 activity inhibitor is a polypeptide, the amino acid sequence of which comprises at least ten consecutive amino acids of a protein selected from the group consisting of noggin, chordin, gremlin, and cerberus 1 homolog

9. The method of claim 1 wherein the bone morphogenetic protein-4 activity inhibitor is a polypeptide the amino acid sequence of which comprises at least ten consecutive amino acids of noggin.

10. The method of claim 9 wherein the amino acid sequence of noggin is selected from the group consisting of SEQ ID NO: 4 and SEQ ID NO: 6.

11. The method of claim 1 wherein the bone morphogenetic protein-4 activity inhibitor is an antibody to bone morphogenetic protein-4.

12. The method of claim 1 wherein the bone morphogenetic protein-4 activity inhibitor is an antisense oligonucleotide that binds to a bone morphogenetic protein-4 nucleic acid sequence.

13. The method of claim 1 wherein the bone morphogenetic protein-4 activity inhibitor is an antisense oligonucleotide that binds to at least a portion of a bone morphogenetic protein-4 nucleic acid sequence.

14. The method of claim 1 wherein the cancer is a carcinoma.

15. The method of claim 14 wherein the carcinoma is selected from the group consisting of bladder cancer, breast cancer, colon cancer, kidney cancer, lung cancer, ovarian cancer, thyroid cancer, endometrial cancer, omental cancer, testicular cancer, and liver cancer.

16. The method of claim 1 wherein the cancer is lung cancer.

17. The method of claim 1 wherein the patient is a human.

18. The method of claim 1 wherein the bone morphogenetic protein-4 activity inhibitor further comprises a pharmaceutically acceptable carrier.

19. The method of claim 18 wherein the bone morphogenetic protein-4 activity inhibitor is administered orally, enterically, intravenously, peritoneally, subcutaneously, transdermally, parenterally, intratumorally, or rectally.

20. A method for the treatment of cancer comprising administering to a patient a therapeutically effective amount of an expression vector having a nucleic acid sequence encoding a bone morphogenetic protein-4 activity inhibitor.

21. The method of claim 20 wherein the expression vector further comprises a selective promoter that is operably linked to the nucleic acid sequence encoding a bone morphogenetic protein-4 activity inhibitor.

22. The method of claim 21 wherein the selective promoter is carcinoembryonic antigen (CEA) promoter.

23. The method of claim 20 wherein the bone morphogenetic protein-4 activity inhibitor is a polypeptide that specifically binds to bone morphogenetic protein-4.

24. The method of claim 20 wherein the bone morphogenetic protein-4 activity inhibitor is a polypeptide that specifically binds to a bone morphogenetic protein-4 receptor.

25. The method of claim 24 wherein the bone morphogenetic protein-4 receptor is bone morphogenetic protein IB receptor.

26. The method of claim 20 wherein the bone morphogenetic protein-4 activity inhibitor is selected from the group consisting of noggin, chordin, gremlin, and cerberus 1 homolog.

27. The method of claim 20 wherein the bone morphogenetic protein-4 activity inhibitor is noggin.

28. The method of claim 27 wherein the amino acid sequence of noggin is selected from the group consisting of SEQ ID NO: 4 and SEQ ID NO: 6.

29. The method of claim 20, wherein the bone morphogenetic protein-4 activity inhibitor is a polypeptide the amino acid sequence of which comprises at least ten consecutive amino acids of noggin.

30. The method of claim 20, wherein the amino acid sequence of noggin is selected from the group consisting of SEQ ID NO: 4 and SEQ ID NO: 6.

31. The method of claim 20 wherein the cancer is a carcinoma.

32. The method of claim 31 wherein the carcinoma is selected from the group consisting of bladder cancer, breast cancer, colon cancer, kidney cancer, lung cancer, ovarian cancer, thyroid cancer, endometrial cancer, omental cancer, testicular cancer, and liver cancer.

33. The method of claim 20 wherein the cancer is lung cancer.

34. The method of claim 20 wherein the patient is a human.

35. The method of claim 20 wherein the expression vector further comprises a pharmaceutically acceptable carrier.

36. The method of claim 35 wherein the expression vector is administered orally, enterically, intravenously, peritoneally, subcutaneously, transdermally, parenterally, intratumorally, or rectally.

37. A method for the treatment of cancer comprising administering to a patient a therapeutically effective amount of an expression vector encoding an antisense oligonucleotide that binds to a bone morphogenetic protein-4 nucleic acid sequence.

38. The method of claim 37 wherein the expression vector further comprises a selective promoter.

39. The method of claim 38 wherein the expression vector is carcinoembryonic antigen (CEA) promoter.

40. The method of claim 37 wherein the cancer is a carcinoma.

41. The method of claim 37 wherein the carcinoma is selected from the group consisting of bladder cancer, breast cancer, colon cancer, kidney cancer, lung cancer, ovarian cancer, thyroid cancer, endometrial cancer, omental cancer, testicular cancer, and liver cancer.

42. The method of claim 41 wherein the cancer is lung cancer.

43. The method of claim 37 wherein the patient is a human.

44. The method of claim 37 wherein the expression vector further comprises a pharmaceutically acceptable carrier.

45. The method of claim 44 wherein the expression vector is administered orally, enterically, intravenously, peritoneally, subcutaneously, transderinally, parenterally, intratumorally, or rectally.

46. An article of manufacture comprising packaging material and, contained within the packaging material, a compound that is a bone morphogenetic protein-4 activity inhibitor, wherein the packaging material indicates that the compound can be used for treating cancer in a patient.

47. The article of manufacture of claim 46 wherein the cancer is a carcinoma.

48. The article of manufacture of claim 47 wherein the carcinoma is selected from the group consisting of bladder cancer, breast cancer, colon cancer, kidney cancer, lung cancer, ovarian cancer, thyroid cancer, endometrial cancer, omental cancer, testicular cancer, and liver cancer.

49. The article of manufacture of claim 46 wherein the cancer is lung cancer.

50. A method for the diagnosis of cancer in a patient, comprising
obtaining a biological sample from a patient and
measuring the level of bone morphogenetic protein-4 in the biological sample, wherein an elevated level of bone morphogenetic protein-4 indicates cancer in the patient.

51. The method of claim 50 wherein the cancer is a carcinoma.

52. The method of claim 51 wherein the carcinoma is selected from the group consisting of bladder cancer, breast cancer, colon cancer, kidney cancer, lung cancer, ovarian cancer, thyroid cancer, endometrial cancer, omental cancer, testicular cancer, and liver cancer.

53. The method of claim 50, wherein the cancer is lung cancer.

54. The method of claim 50 wherein the level of bone morphogenetic protein-4 is measured by an immunoassay.

55. The method of claim 54 wherein the immunoassay is selected from the group consisting of Enzyme Linked immunosorbent Assay (ELISA), Western blot, immunoprecipitation, in situ immunohistochemistry, and immunofluorescence.

56. The method of claim 50 wherein the assay used to measure the level of bone morphogenetic protein-4 is Enzyme-Linked Immunosorbent Assay (ELISA).

57. The method of claim 50, wherein the biological sample is selected from a group consisting of blood, blood serum, urine, sputum, synovial fluid, ascites, and tissue.

58. The method of claim 50 wherein the biological sample is blood serum.

59. A method for the diagnosis of cancer in a patient, which method comprises detecting the overexpression of bone morphogenetic protein-4 in the patient, the overexpression of bone morphogenetic protein-4 indicating the presence of cancer, the method comprising the steps of:
(i) quantifying in vivo or in vitro the presence of bone morphogenetic protein-2 in a patient or a biological sample obtained from a patient;
(ii) comparing the result obtained in step (i) to that of a normal, non-cancerous patient; and
(iii) diagnosing for the presence of cancer based on an increased level of bone morphogenetic protein-4 in step (ii) relative to a normal, non-cancerous patient.

60. The method of claim 59 wherein the cancer is a carcinoma.

61. Me method of claim 60 wherein the carcinoma is selected from the group consisting of bladder cancer, breast cancer, colon cancer, kidney cancer, lung cancer, ovarian cancer, thyroid cancer, endometrial cancer, omental cancer, testicular cancer, and liver cancer.

62. The method of claim 59 wherein the cancer is lung cancer.

63. The method of claim 59 wherein bone morphogenetic protein-4 is quantified by an immunoassay.

64. The method of claim 59 wherein the bone morphogenetic protein-4 is quantified by Enzyme-Linked Immunosorbent Assay (ELISA).


SEQUENCE LISTING (18 sequences)

SEQ ID NO: 1 - DNA, 1547 bp, Homo sapiens
  source (1)..(1547): Homo sapiens  Taxon9606
ggggacttcttgaacttgcagggagaataacttgcgcaccccactttgcgccggtgcctttgccccagcggagcctgcttcgccatctccgagccccaccgcccctccactcctcggccttgcccgacactgagacgctgttcccagcgtgaaaagagagactgcgcggccggcacccgggagaaggaggaggcaaagaaaaggaacggacattcggtccttgcgccaggtcctttgaccagagtttttccatgtggacgctctttcaatggacgtgtccccgcgtgcttcttagacggactgcggtctcctaaaggtcgaccatggtggccgggacccgctgtcttctagcgttgctgcttccccaggtcctcctgggcggcgcggctggcctcgttccggagctgggccgcaggaagttcgcggcggcgtcgtcgggccgcccctcatcccagccctctgacgaggtcctgagcgagttcgagttgcggctgctcagcatgttcggcctgaaacagagacccacccccagcagggacgccgtggtgcccccctacatgctagacctgtatcgcaggcactcaggtcagccgggctcacccgccccagaccaccggttggagagggcagccagccgagccaacactgtgcgcagcttccaccatgaagaatctttggaagaactaccagaaacgagtgggaaaacaacccggagattcttctttaatttaagttctatccccacggaggagtttatcacctcagcagagcttcaggttttccgagaacagatgcaagatgctttaggaaacaatagcagtttccatcaccgaattaatatttatgaaatcataaaacctgcaacagccaactcgaaattccccgtgaccagacttttggacaccaggttggtgaatcagaatgcaagcaggtgggaaagttttgatgtcacccccgctgtgatgcggtggactgcacagggacacgccaaccatggattcgtggtggaagtggcccacttggaggagaaacaaggtgtctccaagagacatgttaggataagcaggtctttgcaccaagatgaacacagctggtcacagataaggccattgctagtaacttttggccatgatggaaaagggcatcctctccacaaaagagaaaaacgtcaagccaaacacaaacagcggaaacgccttaagtccagctgtaagagacaccctttgtacgtggacttcagtgacgtggggtggaatgactggattgtggctcccccggggtatcacgccttttactgccacggagaatgcccttttcctctggctgatcatctgaactccactaatcatgccattgttcagacgttggtcaactctgttaactctaagattcctaaggcatgctgtgtcccgacagaactcagtgctatctcgatgctgtaccttgacgagaatgaaaaggttgtattaaagaactatcaggacatggttgtggagggttgtgggtgtcgctagtacagcaaaattaaatacataaatatatatata

SEQ ID NO: 2 - protein, 396 aa, Homo sapiens
  misc_feature (429)..(1127): Region  TGF-beta propeptide
MVAGTRCLLALLLPQVLLGGAAGLVPELGRRKFAAASSGRPSSQPSDEVLSEFELRLLSMFGLKQRPTPSRDAVVPPYMLDLYRRHSGQPGSPAPDHRLERAASRANTVRSFHHEESLEELPETSGKTTRRFFFNLSSIPTEEFITSAELQVFREQMQDALGNNSSFHHRINIYEIIKPATANSKFPVTRLLDTRLVNQNASRWESFDVTPAVMRWTAQGHANHGFVVEVAHLEEKQGVSKRHVRISRSLHQDEHSWSQIRPLLVTFGHDGKGHPLHKREKRQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 3 - DNA, 699 bp, Homo sapiens
  source (1)..(699): Homo sapiens  Taxon9606
atggagcgctgccccagcctaggggtcaccctctacgccctggtggtggtcctggggctgcgggcgacaccggccggcggccagcactatctccacatccgcccggcacccagcgacaacctgcccctggtggacctcatcgaacacccagaccctatctttgaccccaaggaaaaggatctgaacgagacgctgctgcgctcgctgctcgggggccactacgacccaggcttcatggccacctcgccccccgaggaccggcccggcgggggcgggggtgcagctgggggcgcggaggacctggcggagctggaccagctgctgcggcagcggccgtcgggggccatgccgagcgagatcaaagggctagagttctccgagggcttggcccagggcaagaagcagcgcctaagcaagaagctgcggaggaagttacagatgtggctgtggtcgcagacattctgccccgtgctgtacgcgtggaacgacctgggcagccgcttttggccgcgctacgtgaaggtgggcagctgcttcagtaagcgctcgtgctccgtgcccgagggcatggtgtgcaagccgtccaagtccgtgcacctcacggtgctgcggtggcgctgtcagcggcgcgggggccagcgctgcggctggattcccatccagtaccccatcatttccgagtgcaagtgctcgtgctag

SEQ ID NO: 4 - protein, 232 aa, Homo sapiens
MERCPSLGVTLYALVVVLGLRATPAGGQHYLHIRPAPSDNLPLVDLIEHPDPIFDPKEKDLNETLLRSLLGGHYDPGFMATSPPEDRPGGGGGAAGGAEDLAELDQLLRQRPSGAMPSEIKGLEFSEGLAQGKKQRLSKKLRRKLQMWLWSQTFCPVLYAWNDLGSRFWPRYVKVGSCFSKRSCSVPEGMVCKPSKSVHLTVLRWRCQRRGGQRCGWIPIQYPIISECKCSC

SEQ ID NO: 5 - DNA, 699 bp, Mus musculus
  gene (1)..(699): nog
atggagcgctgccccagcctgggggtcaccctctacgccctggtggtggtcctggggctgcgggcagcaccagccggcggccagcactatctacacatccgcccagcacccagcgacaacctgcccttggtggacctcatcgaacatccagaccctatctttgaccctaaggagaaggatctgaacgagacgctgctgcgctcgctgctcgggggccactacgacccgggctttatggccacttcgcccccagaggaccgacccggagggggcgggggaccggctggaggtgccgaggacctggcggagctggaccagctgctgcggcagcggccgtcgggggccatgccgagcgagatcaaagggctggagttctccgagggcttggcccaaggcaagaaacagcgcctgagcaagaagctgaggaggaagttacagatgtggctgtggtcacagaccttctgcccggtgctgtacgcgtggaatgacctaggcagccgcttttggccacgctacgtgaaggtgggcagctgcttcagcaagcgctcctgctctgtgcccgagggcatggtgtgtaagccatccaagtctgtgcacctcacggtgctgcggtggcgctgtcagcggcgcgggggtcagcgctgcggctggattcccatccagtaccccatcatttccgagtgtaagtgttcctgctag

SEQ ID NO: 6 - protein, 232 aa, Mus musculus
MERCPSLGVTLYALVVVLGLRAAPAGGQHYLHIRPAPSDNLPLVDLIEHPDPIFDPKEKDLNETLLRSLLGGHYDPGFMATSPPEDRPGGGGGPAGGAEDLAELDQLLRQRPSGAMPSEIKGLEFSEGLAQGKKQRLSKKLRRKLQMWLWSQTFCPVLYAWNDLGSRFWPRYVKVGSCFSKRSCSVPEGMVCKPSKSVHLTVLRWRCQRRGGQRCGWIPIQYPIISECKCSC

SEQ ID NO: 7 - DNA, 3547 bp, Homo sapiens
  source (1)..(3547): Taxon9606
cccgggtcagcgcccgcccgcccgcgctcctcccggccgctcctcccgccccgcccggcccggcgccgactctgcggccgcccgacgagcccctcgcggcactgccccggccccggccccggccccggccccctcccgccgcaccgcccccggcccggccctccgccctccgcactcccgcctccctccctccgcccgctcccgcgccctcctccctccctcctccccagctgtcccgttcgcgtcatgccgagcctcccggccccgccggccccgctgctgctcctcgggctgctgctgctcggctcccggccggcccgcggcgccggccccgagccccccgtgctgcccatccgttctgagaaggagccgctgcccgttcggggagcggcaggctgcaccttcggcgggaaggtctatgccttggacgagacgtggcacccggacctaggggagccattcggggtgatgcgctgcgtgctgtgcgcctgcgaggcgcctcagtggggtcgccgtaccaggggccctggcagggtcagctgcaagaacatcaaaccagagtgcccaaccccggcctgtgggcagccgcgccagctgccgggacactgctgccagacctgcccccaggagcgcagcagttcggagcggcagccgagcggcctgtccttcgagtatccgcgggacccggagcatcgcagttatagcgaccgcggggagccaggcgctgaggagcgggcccgtggtgacggccacacggacttcgtggcgctgctgacagggccgaggtcgcaggcggtggcacgagcccgagtctcgctgctgcgctctagcctccgcttctctatctcctacaggcggctggaccgccctaccaggatccgcttctcagactccaatggcagtgtcctgtttgagcaccctgcagcccccacccaagatggcctggtctgtggggtgtggcgggcagtgcctcggttgtctctgcggctccttagggcagaacagctgcatgtggcacttgtgacactcactcacccttcaggggaggtctgggggcctctcatccggcaccgggccctggctgcagagaccttcagtgccatcctgactctagaaggccccccacagcagggcgtagggggcatcaccctgctcactctcagtgacacagaggactccttgcattttttgctgctcttccgagggctgctggaacccaggagtgggggactaacccaggttcccttgaggctccagattctacaccaggggcagctactgcgagaacttcaggccaatgtctcagcccaggaaccaggctttgctgaggtgctgcccaacctgacagtccaggagatggactggctggtgctgggggagctgcagatggccctggagtgggcaggcaggccagggctgcgcatcagtggacacattgctgccaggaagagctgcgacgtcctgcaaagtgtcctttgtggggctgatgccctgatcccagtccagacgggtgctgccggctcagccagcctcacgctgctaggaaatggctccctgatctatcaggtgcaagtggtagggacaagcagtgaggtggtggccatgacactggagaccaagcctcagcggagggatcagcgcactgtcctgtgccacatggctggactccagccaggaggacacacggccgtgggtatctgccctgggctgggtgcccgaggggctcatatgctgctgcagaatgagctcttcctgaatgtgggcaccaaggacttcccagacggagagcttcgggggcacgtggctgccctgccctactgtgggcatagcgcccgccatgacacgctgcccgtgcccctagcaggagccctggtgctaccccctgtgaagagccaagcagcagggcacgcctggctttccttggatacccactgtcacctgcactatgaagtgctgctggctgggcttggtggctcagaacaaggcactgtcactgcccacctccttgggcctcctggaacgccagggcctcggcggctgctgaagggattctatggctcagaggcccagggtgtggtgaaggacctggagccggaactgctgcggcacctggcaaaaggcatggcctccctgctgatcaccaccaagggtagccccagaggggagctccgagggcaggtgcacatagccaaccaatgtgaggttggcggactgcgcctggaggcggccggggccgagggggtgcgggcgctgggggctccggatacagcctctgctgcgccgcctgtggtgcctggtctcccggccctagcgcccgccaaacctggtggtcctgggcggccccgagaccccaacacatgcttcttcgaggggcagcagcgcccccacggggctcgctgggcgcccaactacgacccgctctgctcactctgcacctgccagagacgaacggtgatctgtgacccggtggtgtgcccaccgcccagctgcccacacccggtgcaggctcccgaccagtgctgccctgtttgccctgagaaacaagatgtcagagacttgccagggctgccaaggagccgggacccaggagagggctgctattttgatggtgaccggagctggcgggcagcgggtacgcggtggcaccccgttgtgcccccctttggcttaattaagtgtgctgtctgcacctgcaaggggggcactggagaggtgcactgtgagaaggtgcagtgtccccggctggcctgtgcccagcctgtgcgtgtcaaccccaccgactgctgcaaacagtgtccagtggggtcgggggcccacccccagctgggggaccccatgcaggctgatgggccccggggctgccgttttgctgggcagtggttcccagagagtcagagctggcacccctcagtgcccccttttggagagatgagctgtatcacctgcagatgtggggcaggggtgcctcactgtgagcgggatgactgttcactgccactgtcctgtggctcggggaaggagagtcgatgctgttcccgctgcacggcccaccggcggccagccccagagaccagaactgatccagagctggagaaagaagccgaaggctcttagggagcagccagagggccaagtgaccaagaggatggggcctgagctggggaaggggtggcatcgaggaccttcttgcattctcctgtgggaagcccagtgcctttgctcctctgtcctgcctctactcccacccccactacctctgggaaccacagctccacaagggggagaggcagctgggccagaccgaggtcacagccactccaagtcctgccctgccaccctcggcctctgtcctggaagccccacccctttcctcctgtacataatgtcactggcttgttgggatttttaatttatcttcactcagcaccaagggcccccgacactccactcctgctgcccctgagctgagcagagtcattattggagagttttgtatttattaaaacatttctttttcagtcaaaaaaaaaaaaaaaaaaaaaaaaaa

SEQ ID NO: 8 - protein, 955 aa, Homo sapiens [sequence omitted]

SEQ ID NO: 9 - DNA, 3299 bp, Homo sapiens
  source (1)..(3299): small intestine
ataataattaggccaagcgttgaatagtacggggggggggggggggcgagccccggcggctctggccgcggccgcactcagcgccacgcgtcgaaagcgcaggccccgaggacccgccgcactgacagtatgagccgcacagcctacacggtgggagccctgcttctcctcttggggaccctgctgccggctgctgaagggaaaaagaaagggtcccaaggtgccatccccccgccagacaaggcccagcacaatgactcagagcagactcagtcgccccagcagcctggctccaggaaccgggggcggggccaagggcggggcactgccatgcccggggaggaggtgctggagtccagccaagaggccctgcatgtgacggagcgcaaatacctgaagcgagactggtgcaaaacccagccgcttaagcagaccatccacgaggaaggctgcaacagtcgcaccatcatcaaccgcttctgttacggccagtgcaactctttctacatccccaggcacatccggaaggaggaaggttcctttcagtcctgctccttctgcaagcccaagaaattcactaccatgatggtcacactcaactgccctgaactacagccacctaccaagaagaagagagtcacacgtgtgaagcagtgtcgttgcatatccatcgatttggattaagccaaatccaggtgcacccagcatgtcctaggaatgcagacccaggaagtcccagacctaaaacaaccagattcttacttggcttaaacctagaggccagaagaacccccagctgcctcctggcaggagcctgcttgtgcgtagttcgtgtgcatgagtgtggatgggtgcctgtgggtgtttttagacaccagagaaaacacagtctctgctagagagcacttcctattttgtaaacctatctgctttaatggggatgtaccagaaacccacctcaccccggctcacatctaaaggggcggggccgtggtctggttctgactttgtgtttttgtgccctcctggggaccagaatctcctttcggaatgaatgttcatggaagaggctcctctgagggcaagagacctgttttagtgctgcattcgacatggaaaagtccttttaacctgtgcttgcatcctcctttcctcctcctcctcacaatccatctcttcttaagttgacagtgactatgtcagtctaatctcttgtttgccagggttcctaaattaattcacttaaccatgatgcaaatgtttttcatttggtgaagacctccagactctgggagaggctggtgtgggcaaggacaagcaggatagtggagtgagaaagggagggtggagggtgaggccaaatcaggtccagcaaaagtcagtagggacattgcagaagcttgaaaggccaataccagaacacaggctgatgcttctgagaaagtcttttcctagtatttaacaaaacccaagtgaacagaggagaaatgagattgccagaaagtgattaactttggccgttgcaatctgctcaaacctaacaccaaactgaaaacataaatactgaccactcctatgttcggacccaagcaagttagctaaaccaaaccaactcctctgctttgtccctcaggtggaaaagagaggtagtttagaactctctgcataggggtgggaattaatcaaaaacctcagaggctgaaattcctaatacctttcctttatcgtggttatagtcagctcatttccattccactatttcccataatgcttctgagagccactaacttgattgataaagatcctgcctctgctgagtgtacctgacagtagtctaagatgagagagtttagggactactctgttttaacaagaaatattttgggggtctttttgttttaactattgtcaggagattgggctaaagagaagacgacgagagtaaggaaataaagggaattgcctctggctagagagtagttaggtgttaatacctggtagagatgtaagggatatgacctccctttctttatgtgctcacttgaggatctgaggggaccctgttaggagagcatagcatcatgatgtattagctgttcatctgctactggttggatggacataactattgtaactattcagtatttactggtaggcactgtcctctgattaaacttggcctactggcaatggctacttaggattgatctaagggccaaagtgcagggtgggtgaactttattgtactttggatttggttaacctgttttcctcaagcctgaggttttatatacaaactccctgaatactctttttgccttgttacttctcagcctcctagccaagtcctatgtaatatggaaaacaaacactgcagacttgagattcagttgccgatcaaggctctggcattcagagaacccttgcaactcgagaagctgtttttgatttcgtttttgttttgaaccggtgctctcccatctaacaactaacaaggaccatttccaggcgggagatattttaaacacccaaaatgttgggtctgatttccaaacttttaaactcactactgatgattctcacgctaggcgaatttgtccaaacacatagtgtgtgtgttttgtatacactgtatgaccccaccccaaatctttgtattgtccacattctccaacaataaagcacagagtggatttaattaagcacacaaatgctaaggcagaattttgagggtgggagagaagaaaagggaaagaagctgaaaatgtaaaaccacaccagggaggaaaaatgacattcagaaccaccaaacactgaatttctcttgttgttttaactctcccacaagaatgcaatttcgttaatggagatgacttaagttggcagcagtaatcttcttttaggagcttgtaccacagtcttgcacataagtgcagatttgccccaagtaaagagaatttcctcaacactaacttcacggggataatcaccacgtaactacccttaaagcatatcactagccaaagaggggaatatctgttcttcttactgtgcctatattaagactagtacaaatgtggtgtgtcttccaactttcattgaaaatgccatatctataccatattttattcgagtcactgatgatgtaatgatatattttttcattattatagtagaatatttttatggcaagagatttgtggtcttgatcatacctattaaaataatgccaaacaccaaatatgaattttatgatgtacactttgtgcttggcattaaaagaaaaaaacacacacgcc

SEQ ID NO: 10 - protein, 184 aa, Homo sapiens
MSRTAYTVGALLLLLGTLLPAAEGKKKGSQGAIPPPDKAQHNDSEQTQSPQQPGSRNRGRGQGRGTAMPGEEVLESSQEALHVTERKYLKRDWCKTQPLKQTIHEEGCNSRTIINRFCYGQCNSFYIPRHIRKEEGSFQSCSFCKPKKFTTMMVTLNCPELQPPTKKKRVTRVKQCRCISIDLD

SEQ ID NO: 11 - DNA, 804 bp, Homo sapiens
  source (1)..(804)
atgcatctcctcttatttcagctgctggtactcctgcctctaggaaagaccacacggcaccaggatggccgccagaatcagagttctctttcccccgtactcctgccaaggaatcaaagagagcttcccacaggcaaccatgaggaagctgaggagaagccagatctgtttgtcgcagtgccacaccttgtagccaccagccctgcaggggaaggccagaggcagagagagaagatgctgtccagatttggcaggttctggaagaagcctgagagagaaatgcatccatccagggactcagatagtgagcccttcccacctgggacccagtccctcatccagccgatagatggaatgaaaatggagaaatctcctcttcgggaagaagccaagaaattctggcaccacttcatgttcagaaaaactccggcttctcagggggtcatcttgcccatcaaaagccatgaagtacattgggagacctgcaggacagtgcccttcagccagactataacccacgaaggctgtgaaaaagtagttgttcagaacaacctttgctttgggaaatgcgggtctgttcattttcctggagccgcgcagcactcccatacctcctgctctcactgtttgcctgccaagttcaccacgatgcacttgccactgaactgcactgaactttcctccgtgatcaaggtggtgatgctggtggaggagtgccagtgcaaggtgaagacggagcatgaagatggacacatcctacatgctggctcccaggattcctttatcccaggagtttcagcttga

SEQ ID NO: 12 - protein, 267 aa, Homo sapiens
  misc_feature (361)..(741): DAN domain
MHLLLFQLLVLLPLGKTTRHQDGRQNQSSLSPVLLPRNQRELPTGNHEEAEEKPDLFVAVPHLVATSPAGEGQRQREKMLSRFGRFWKKPEREMHPSRDSDSEPFPPGTQSLIQPIDGMKMEKSPLREEAKKFWHHFMFRKTPASQGVILPIKSHEVHWETCRTVPFSQTITHEGCEKVVVQNNLCFGKCGSVHFPGAAQHSHTSCSHCLPAKFTTMHLPLNCTELSSVIKVVMLVEECQCKVKTEHEDGHILHAGSQDSFIPGVSA

SEQ ID NO: 13 - DNA, 2032 bp, Homo sapiens
  source (1)..(2032): Homo sapiens  Taxon9606
cgcggggcgcggagtcggcggggcctcgcgggacgcgggcagtgcggagaccgcggcgctgaggacgcgggagccgggagcgcacgcgcggggtggagttcagcctactctttcttagatgtgaaaggaaaggaagatcatttcatgccttgttgataaaggttcagacttctgctgattcataaccatttggctctgagctatgacaagagaggaaacaaaaagttaaacttacaagcctgccataagtgagaagcaaacttccttgataacatgcttttgcgaagtgcaggaaaattaaatgtgggcaccaagaaagaggatggtgagagtacagcccccaccccccgtccaaaggtcttgcgttgtaaatgccaccaccattgtccagaagactcagtcaacaatatttgcagcacagacggatattgtttcacgatgatagaagaggatgactctgggttgcctgtggtcacttctggttgcctaggactagaaggctcagattttcagtgtcgggacactcccattcctcatcaaagaagatcaattgaatgctgcacagaaaggaacgaatgtaataaagacctacaccctacactgcctccattgaaaaacagagattttgttgatggacctatacaccacagggctttacttatatctgtgactgtctgtagtttgctcttggtccttatcatattattttgttacttccggtataaaagacaagaaaccagacctcgatacagcattgggttagaacaggatgaaacttacattcctcctggagaatccctgagagacttaattgagcagtctcagagctcaggaagtggatcaggcctccctctgctggtccaaaggactatagctaagcagattcagatggtgaaacagattggaaaaggtcgctatggggaagtttggatgggaaagtggcgtggcgaaaaggtagctgtgaaagtgttcttcaccacagaggaagccagctggttcagagagacagaaatatatcagacagtgttgatgaggcatgaaaacattttgggtttcattgctgcagatatcaaagggacagggtcctggacccagttgtacctaatcacagactatcatgaaaatggttccctttatgattatctgaagtccaccaccctagacgctaaatcaatgctgaagttagcctactcttctgtcagtggcttatgtcatttacacacagaaatctttagtactcaaggcaaaccagcaattgcccatcgagatctgaaaagtaaaaacattctggtgaagaaaaatggaacttgctgtattgctgacctgggcctggctgttaaatttattagtgatacaaatgaagttgacataccacctaacactcgagttggcaccaaacgctatatgcctccagaagtgttggacgagagcttgaacagaaatcacttccagtcttacatcatggctgacatgtatagttttggcctcatcctttgggaggttgctaggagatgtgtatcaggaggtatagtggaagaataccagcttccttatcatgacctagtgcccagtgacccctcttatgaggacatgagggagattgtgtgcatcaagaagttacgcccctcattcccaaaccggtggagcagtgatgagtgtctaaggcagatgggaaaactcatgacagaatgctgggctcacaatcctgcatcaaggctgacagccctgcgggttaagaaaacacttgccaaaatgtcagagtcccaggacattaaactctgataggagaggaaaagtaagcatctctgcagaaagccaacaggtactcttctgtttgtgggcagagcaaaagacatcaaataagcatccacagtacaagccttgaacatcgtcctgcttcccagtgggttcagacctcacctttcagggagcgacctgggcaaagacagagaagctcccagaaggagagattgatccgtgtctgtttgtaggcggagaaaccgttgggtaacttgttcaagatatgatgcat

SEQ ID NO: 14 - protein, 502 aa, Homo sapiens
  misc_feature (367)..(606): Activin_recp; Region  Activin types I and II
MLLRSAGKLNVGTKKEDGESTAPTPRPKVLRCKCHHHCPEDSVNNICSTDGYCFTMIEEDDSGLPVVTSGCLGLEGSDFQCRDTPIPHQRRSIECCTERNECNKDLHPTLPPLKNRDFVDGPIHHRALLISVTVCSLLLVLIILFCYFRYKRQETRPRYSIGLEQDETYIPPGESLRDLIEQSQSSGSGSGLPLLVQRTIAKQIQMVKQIGKGRYGEVWMGKWRGEKVAVKVFFTTEEASWFRETEIYQTVLMRHENILGFIAADIKGTGSWTQLYLITDYHENGSLYDYLKSTTLDAKSMLKLAYSSVSGLCHLHTEIFSTQGKPAIAHRDLKSKNILVKKNGTCCIADLGLAVKFISDTNEVDIPPNTRVGTKRYMPPEVLDESLNRNHFQSYIMADMYSFGLILWEVARRCVSGGIVEEYQLPYHDLVPSDPSYEDMREIVCIKKLRPSFPNRWSSDECLRQMGKLMTECWAHNPASRLTALRVKKTLAKMSESQDIKL

SEQ ID NO: 15 - DNA, 20 bp, Homo sapiens
acgagagctctcactggtcc

SEQ ID NO: 16 - DNA, 20 bp, Homo sapiens
cattccggattacatgaggg

SEQ ID NO: 17 - DNA, 11233 bp, Homo sapiens
  misc_feature (1)..(3165): 5′ flanking region
gaattccttccgtagcttcaccagacacctaattggccaagaaggtttgaagacctgatgtggttcttaattggggatggggaattaagggctactgtatctataggattatcttttcacttgcatagacctatttggtgtgttcagggcatagtgatactataattgccatatttaacagtttataaagttcaagcccagcatattctttgcctgtttaatgatgtcttggtatcagccttttaatggtacttatcagcatagaaaatggaaacaaaataacttttaaaacagtagctctcaagctttagtgtgctcagaatgaccagagaaccttgtgaaatatacagatttctgggtccagatctggggcaggaccaggaagtctgcatttcatctgcacccccaccctactctgaggcttatagtcctgagaacatgctttgaaaaaggctgtcccaagggctcgcagacaggctattgaccagctactctttcttgatgttctccaggaaaaacccaacaaaggaatgcctttcattgagtagtagcagcataggagcaatagttgctcctgaattatggtgggtttcccctcttcatcaatgtgctttaagggtacagtttcatttggtctatctaccatgttctataaaaacatgaaaattcacaggtaagtttgagatacagaaaataactaaactgattcttctcacgaactctgatcactaggctgtggttgatttagctctctaaccaacaagtaatttgttctttggcatgagtaaggggggaaaaggaggagtgggtaaaagcagctgataacagatggcttgcgcccatctaaaatgtggggagagaaataaagctgtcccaagagaactaaagctgagttctctcgtcatatatctgaagattcatatcaggggtctaaacatggtatgtcgggtagcttaattggaaactcctggactgtgagtgtcacagactcatggatgggccaatcagtggccactttagtgtctgggctgcagcaaaatgagacaatagctgtcattcacaaacctttggaattaaaaaaaccccgaaatgacattggtgctttaaagtaaaataaagtcctgcctttaagtccagcatatcactgttgtttctgagtttaaatattaagaaccacatttcgttaatgattaaaacaacagtgattgatttaggggctcagtgagcatttaatctgtcctgacttcaggtaccatgctaaaggagcacaatgcctgatgctgcaggagaaacattaggtaactatttaatggagttttaattttctgttattatttttaataattaattgtgattttgactatttggaagctacaggtatattttgtcctccttttggggtggtgttattgccctgccctgttttaatcagtggttcttagagaaagtgaactcaggagtgacttaaaatgaaggaagacggactttggctaaaattacaattaaataatcaaatcattttcaaatataaagggagcatgcagatgatctggcccaatcctttcattctgcagatgagaaaactgagactcataggaatgaaaagacttgcccaaagccatacagcttgtttctgttgtttggtgcattaggccaaaagacctaggcctaatagatggaaaagatggcaggatgtcttggccttgctctgacagttgcttctctgatctcagatatttcccaccctttgtaatctgtgttccacacaggaagtagttcttgttttttaaatatcgaaggtgtataaacgtaaagtttttatagatgagccacccagggccaatatctgtttaagtaaagacctaaatgctttgcagagacagtaaagtgtcatgtctgtcccagggaaagaaatccaggacaggaaatgctcagtcttccagcactcctctggctacctggagctcaggctatgagcctcaacccctccctgaagcattagctctggagcagaggctgtgatttacttcagagatctgggcaagtccctttaacctggtagtccttcctttccttgtttgtaaaacagagagatgaggctgatagctccctcacagctccatcagaggcagtgtgtgaaattagttcctgtttgggaaggtttaaaagccaccacattccacctccctgctaatatgattactaaaatgtttttatatgaaagggccaattcctcatctcccctcttcctttaaaaacagaccaaggggcatcttttcttgtctccctgtggcctaaaaggttactgcttctgtggttatctccttggaaagacagagtgtcaggactcttaggtacaccaaaaatgaacaaaaaaatcaacaacaaccataacaccaacaaaaataactgctgtgtcggttcttaagacggcttctgagctagaaacagatttttctaactgtaaaaaacgtggccccagcctgtctgcaggccacctctgtctttaggccttggggggaggagggaagtgagctcatttactggggtctacctcagggtcatcaccaaggtgttctacaaaacgcactttaagaatgttttggaaggaaattcaccttttaacagcccaagaggtatctctctctggcacacagttctgcacacagcctgtttctcaacgtttggaaatcttttaacagtttatggaaggccaccttttaaaccgatccaacagctcctttctccataacctgattttagaggtgtttcattatctctaattactcagggtaaatggtgattactcagtgttttaatcatcagtttgggcagcagttacactaaactcagggaagcccagactcccatgggtatttttggaaggtacggcgactagtcggtgcatgctttctagtacctccgcacgtggtccccaggtgagccccagccgcttcccagagctggaggcagcggcgtcccagctccgacggcagctgcggactcgggcgctgcctgggcttccgggacccgggcctgctaggcgaggtcgggcggctggaggggaggatgtgggcggggctcccatccccagaaagggaggcgagcgagggaggagggaaggagggaggggccgccggggaagaggaggaggaaggaaagaaagaaagcgagggagggaaagaggaggaaggaagatgcgagaaggcagaggaggagggagggagggaaggagcgcggagcccggcccggaagctaggtgagtgtggcatccgagctgagggacgcgagcctgagacgccgctgctgctccggctgagtatctagcttgtctccccgatgggattcccgtccaagctatctcgagcctgcagcgccacagtccccggccctcgcccaggttcactgcaaccgttcagaggtccccaggagctgctgctggcgagcccgctactgcagggacctatggtgagcaaggctacctggtgaggggagacaggcagagggggtctaggagcctccttggggggaagaagctggtcacaggctgtgaccgaggcaaaaggtggcctaattattttccaatagtggtgctggaggtggggatgctggcgctgaaagacctttaaatatcggctactgcccctgcccaggccttctctgtccagcagtccctgggagattctcacctttgggaagtgcggggcaggagagcagaaacaagagaagcccttggtaggggggtcgttgggaaaaactgtggggtcttgggctgaacgcgttgcccacgggctggaggttgcgatccccggacggaaagcgcgggaggaggaaggagagaaccggctctgaggtccagagagagtgagggggcagagcgacggcgagatggggagagaacacctagctggagcaggttctgcggtagagagcgcagtcctgctggcctctggagagtgcgcgccgctacggaggctgcgtcgaggggagtgtcacccaatctggcccccagctggcggggcgccctgagagcttgcgaactgcagttgcaggacgcgccttctccacgagctattttcgtcgacttgcggaacccaaggaacctcgcctctatcatttcacggtgtagggtccctagagacgacagccaagatcccaggggctcccaggacgcttgttcctgcggtgtcgtgtcctatggggagttcctggcgggacgaaaggcggacgcgcggctcttcctggccctccaggcccggaaccgacgggaaaggttcccgtgattcccgagtccctgcaggcttcttccagcgggagttggtccgggggccttagaggcctccaagcactgctttggaggatggtttccaaggatcgcggtttgtgagttgaaggctttgtgagaggttaaacccccaaaagatacatacttggtaaactgaggctacctgtaaacacatttcggcattaggagaagattcgagtagggaagtgaaggacaaccaccccgagttacattcctttcccccaataaaaagctctggggatgaaagttcttttggcttttatcttttcgatttaaaaatttgagaagaaaaatgtgactagagatgaatcctggtgaatccgaaattgaaacacaactcccccttccccttcctatcctctcggttttagaaccgcgctctcccgccccaggagattccttggggccgagggttttccggggaacccgggcgcccgccccttctactgtccctttgccccgcgggcacagcttgcctccgtctgctttctctacttctggacctctcctcgccgggctttttaaagggcttctgcgtctcaaaacaaaacaaaaaaaccctttgctcttcccaaccctttcgcagcccgccccagcggtggcgcgggaccagcaaaggcgaaagccgcgcggctcttgccgggcgcggacggtcgcgcaggggcgcccgcggcctccgcacccggacctgaggtgttggtcgactccgggcatccacggtcgggagggagggctgagctgttcgatcctttacttttcttcctcaaagtctacctgccaatgcccctaagaagaaaaccaagtatgtgcgtggagagtggggcggcaggcaacccgagttcttgagctccggagcgacccaaagcagcaactgggaacagcctcaggaaagggaggtcgggtggagtgggctttggggcaggagtcatggggcccgggccccggggacgacctggcgctcccggccctgctgaacgctgagttgcgcctagtcgggttttcgaagaggcccttgcgcagagcgacccacgcgcgcggcagcatcttcgattagtcaggacatcccagtaactgcttgaactgtaggtaggtaaaattcttgaaggagtatttgctgcgtgcgactctgctgctggtgcaacggaggaagggggtgggggaaggaagtggcgggggaaggagtgtggtggtggtttaaaaaataagggaagccgaggcgagagagacgcagacgcagaggtcgagcgcaggccgaaagctgttcaccgttttctcgactccggggaacatggtgggatttcctttctgcgccgggtcgggagttgtaaaacctcggccacattaagatctgaaaactgtgatgcgtcctttctgcagagacgcctctttctgaatctgcccggagcttcgagccccggcgtctgtccctcagcctggcatggcttcttcgggggtctgctttgcatggggagaggggccacgcagcggcggactaggtttggggattctcggtaatggacccggagcaatgactaacagccgctccctctcactttcccacagcgatcaccctctaacaccctccctcccattcccggccccgcgcgtgacaaggtcggctgctttcagccgggagctagatcggtggcccggctcttcggagccttagcaggcgttcgccaaggggtgactggctgtcattgggagcaatatttggccttgaggagaccctggggaggaagtggcggggagctcgtgtttgcttgtgtgtgtgtgggggggggggtgtgtgtacacgcgcgtgggcagggtccctctgcgctttcctttttaagtgcctctcggtggtgaggctttgggcgggtgagactttcccgacctcgctcccggccccacttaagccgggttcgagctgggagacgcagtcccttcagtgcgccccaaatcctctggcttcaggtggcccggcgcgggggcccagcacgacgcaccgcgccgagaaccgggttctccgtgcgctgcgccagtagccctgggagcgcggcggccgcggggcaccggccgagggctctgccgagcgccgccgggagctcctcccggaccgctgaggctcgggcggcgggcgcggaggttggcctcgcctggaggggcgggcccgcgaggggcggggggctgtggaggaggggagggcgcgcaggccctttcgccgcctgccgcgggaggggcctcggcgctcacgtgactccgaggggctggaagaaaaacagagcctgtctgcggtggagtctcattatattcaaatattccttttaggagccattccgtagtgccatcccgagcaacgcactgctgcagcttccctgagcctttccagcaagtttgttcaagattggctgtcaagaatcatggactgttattatatgccttgttttctgtcagtgagtagacacctcttccttccccctctccggaattcactctgccctcaccacccctgctcgccggctgtcccttccgtcggacctcctttacaatatccacactctgctccctggcagcactgtcgctcccttcttggcccggcagccggggcgctggaagcgtacgggttccttttaaagtgctgctagcgcgcactcgccctctcagcgttgcaagaaaggggagcgcgagggagctaaagagatgaaagcccggggttgtaccttgagggctaaccactcccttcccctatccaacttgtctgggagagcccccagtgtctccgtggcgcgttcccactctcttgtcaaaactcacagaggtctctccggaatcgtctctcaccccttccctggggatgagcgggcacgatcaggcacttttggctgaatatttcaaactcatcggccacaataaaataagccctcaagccacccggttagctcccagaccaccttctcggcttctggaccctgtcgccctctgtcttcgcccagcccctgcctctcactttccctccctctggctctgaaccaactggaagttgtgaaagttgggctctgagggtggaggaaaagggagagaagctgaaggtctaaagtggagagcaatgccattttaattctccctcccccaccccttttcaccccctcaatgttaactgtttatccttcaagaagccacgctgagatcatggcccagatagcagttaggacaaaaaaagattaacaggatggaggctatctgatttggggttatttgactgtaaacaagttagaccaagtaattacagggcaattcttactttcaggccgtgcatggctgcagctggtgggtgggcgggtggtgtgagggagaagacacaaacttgatctttctgacctgctttccatcttgcccctccatttctagccctaaatgcatatgcagacacatctctatttctccctatttattggtgtttgtttattctttaaccttccactcccctccccctccccagagacaccatgattcctggtaaccgaatgctgatggtcgttttattatgccaagtcctgctaggaggcgcgagccatgctagtttgatacctgagacggggaagaaaaaagtcgccgagattcagggccacgcgggaggacgccgctcagggcagagccatgagctcctgcgggacttcgaggcgacacttctgcagatgtttgggctgcgccgccgcccgcagcctagcaagagtgccgtcattccggactacatgcgggatctttaccggcttcagtctggggaggaggaggaagagcagatccacagcactggtcttgagtatcctgagcgcccggccagccgggccaacaccgtgaggagcttccaccacgaaggtcagtctcttcccccagtctgcgtgggggagggctggtgggactggctagaggggcagtgaaagccctggggaagaagagttcgggttacatcaaaccccagtccaggaggctgaggaacagagctgcttacctccaagaatttgcagagctgccgccgaacttattttttggagacagagggggaggtgttcaggggaaggggaatgacagcactcagacgtgggctagccccagcggtgtgtttttgctatatcaaagccttttctgctaggttttctgcccgtttttttcaaagcacctactgaatttaatattacagctgtgtgtttgtcgggtttattcaataggggccttgtaatccgatctgaatgtttcctagcggatgtttcttttccaaagtaaatctgagttattaatccaccagcatcattactgtgttggaatttattttcccctctgtaacatgatcaacaaggcatgctctgtgtttccaagatcgctggggaaatgtttagtaacatactcaatagtggaagagggagagggtggttgtctccatgtttcctcctgcctgtgctctgttggcccctctttttctttacaaccacttgtaaagaaaactgtggacacaaagccaaggtggggggtttaaaagaggagtctgattgtggtgccatagaggagttgacacatagaaattattagacatatcaaggaggctggatatagtttctgtctttggtgcttgagaaatgctagctacattttgctggtttgttagctgccccacttatctgctccttcaaattaaggggtatgcttattttcccccagtaggtttcccctgcataagcagaattcaccattcattgcccaaccctgagctatctcttgactcttccatctttgaaaaaagttcatatgctttttcttttccccttccttcctaactgtgcctagaacatctggagaacatcccagggaccagtgaaaactctgcttttcgtttcctctttaacctcagcagcatccctgagaacgaggcgatctcctctgcagagcttcggctcttccgggagcaggtggaccagggccctgattgggaaaggggcttccaccgtataaacatttatgaggttatgaagcccccagcagaagtggtgcctgggcacctcatcacacgactactggacacgagactggtccaccacaatgtgacacggtgggaaacttttgatgtgagccctgcggtccttcgctggacccgggagaagcagccaaactatgggctagccattgaggtgactcacctccatcagactcggacccaccagggccagcatgtcaggattagccgatcgttacctcaagggagtgggaattgggcccagctccggcccctcctggtcacctttggccatgatggccggggccatgccttgacccgacgccggagggccaagcgtagccctaagcatcactcacagcgggccaggaagaagaataagaactgccggcgccactcgctctatgtggacttcagcgatgtgggctggaatgactggattgtggccccaccaggctaccaggccttctactgccatggggactgcccctttccactggctgaccacctcaactcaaccaaccatgccattgtgcagaccctggtcaattctgtcaattccagtatccccaaagcctgttgtgtgcccactgaactgagtgccatctccatgctgtacctggatgagtatgataaggtggtactgaaaaattatcaggagatggtagtagagggatgtgggtgccgctgagatcaggcagtccttgaggatagacagatatacacaccacacacacacaccacatacaccacacacacacgttcccatccactcacccacacactacacagactgcttccttatagctggacttttatttaaaaaaaaaaaaaaaaaaatggaaaaaatccctaaacattcaccttgaccttatttatgactttacgtgcaaatgttttgaccatattgatcatatattttgacaaaatatatttataactacgtattaaaagaaaaaaataaaatgagtcattattttaaaggtaaatcatgatttttttttctccttaatcctttctcttttccttcgggctcatctcttttgaatgaggcttttttctgttcaggtgagttggaggctggatggaagtcaaaaggtggtacctggaggtggttaagttgtagggacaggaagtaaactgttggcagagagagatggtaattgccagcatgaattgttttctatttctatttaatgttaacaaggatgcagtatcctctcccatctggatgacacatgccttggagaaacactgggatgaaaggagtgtaggtcagattaaagacttcatttcaggccccttgtacatcttctgtttcactcacctgttgaggtgtatcacagctgagcgtgatgaggtctcaaccctagaaaaatgatacccacctctgctttcatgatacctcagggtatctccagttattacaggtaccaatgtgatatttccaaatcaaaactaatttgtacactaacatcataatgtgtgtgtgaaggcatgtttttaaacttattttttttttctccaggtaggactcttttgttttttcttttgtctttttttttttgaaacaagttctctctttgttgccccaggctggtcttgaactcctgggctcaagcaatcttctcatttcggcctctttgggattacaggcatgcactgctattttgtcttttttttttttttgtaacaaataatgtaccctaccttcaaaaagtttgatgactactgttttaatatgccacttgatagaatttcccattgtttcttgactttttcccttgtcctcttttcccaatgtgaaggccttcatcaagtttaggatcccaacagattgggctgggtgggggttgacaatggggtcagatactaaagggtcagaatttctaagcaggcactgtgaaggtgtcccactattatacagaaatctcgag

SEQ ID NO: 18 - protein, 408 aa, Homo sapiens
MIPGNRMLMVVLLCQVLLGGASHASLIPETGKKKVAEIQGHAGGRRSGQSHELLRDFEATLLQMFGLRRRPQPSKSAVIPDYMRDLYRLQSGEEEEEQIHSTGLEYPERPASRANTVRSFHHEEHLENIPGTSENSAFRFLFNLSSIPENEAISSAELRLFREQVDQGPDWERGFHRINIYEVMKPPAEVVPGHLITRLLDTRLVHHNVTRWETFDVSPAVLRWTREKQPNYGLAIEVTHLHQTRTHQGQHVRISRSLPQGSGNWAQLRPLLVTFGHDGRGHALTRRRRAKRSPKHHSQRARKKNKNCRRHSLYVDFSDVGWNDWIVAPPGYQAFYCHGDCPFPLADHLNSTNHAIVQTLVNSVNSSIPKACCVPTELSAISMLYLDEYDKVVLKNYQEMVVEGCGCR